(19) United States
(12) Patent Application Publication
(10) Pub. No.: US 2024/0050335 A1
(43) Pub. Date: Feb. 15, 2024

(54) A SYNERGISTIC PERFUMING COMPOSITION

(71) Applicant: Firmenich SA, Satigny (CH)

(72) Inventor: Sabine VON TSCHARNER, Satigny (CH)

(21) Appl. No.: 18/260,297

(22) PCT Filed: Dec. 16, 2021
(86) PCT No.: PCT/EP2021/086232

§ 371 Date: Jul. 3, 2023

Related U.S. Application Data
(60) Provisional application No. 63/127,235, filed on Dec. 18, 2020.

(30) Foreign Application Priority Data
Feb. 5, 2021 (EP) 21155614.7

(51) Int. Cl. A61K 8/33; A61K 8/49; A61K 8/37; A61K 8/35; A61K 8/92; A61K 8/31; A61K 8/40; A61Q 5/02; A61Q 5/12; A61Q 5/06; A61Q 15/00; A61Q 19/00; C11D 3/50; C11D 11/00; C11D 3/00

(52) U.S. Cl. CPC A61K 8/33; A61K 8/4973; A61K 8/37; A61K 8/35; A61K 8/922; A61K 8/31; A61K 8/40; A61K 8/498; A61Q 5/02; A61Q 5/12; A61Q 5/06; A61Q 15/00; A61Q 19/00; C11D 3/50; C11D 11/0023; C11D 3/001

(57) ABSTRACT

The present invention relates to the field of perfumery. More particularly, it concerns perfuming composition of matter as defined above. Following what is mentioned herein, the invention's perfuming composition matter as a part of a perfuming composition or a perfumed consumer product and the use of the invention's perfuming composition are also objects of the invention.

### TECHNICAL FIELD

The present invention relates to the field of perfumery. More particularly, it concerns perfuming composition of matter as defined above. Following what is mentioned herein, the invention's perfuming composition matter as a part of a perfuming composition or a perfumed consumer product and the use of the invention's perfuming composition are also objects of the invention.

### BACKGROUND OF THE INVENTION

The perfume industry has a particular interest for compositions or additives which are capable of prolonging or enhancing the perfuming effect for a certain period of time. It is particularly desirable to obtain long-lasting properties in order to enhance the consumer experience Long-lasting perfumes are desirable for various applications, as for example fine or functional perfumery or cosmetic preparations. The washing and softening of textiles are particular fields in which there is a constant need to enable the effect of active substances, in particular perfumes, or perfuming compositions, to be effective for a certain period of time after washing, softening and drying. Indeed, many active substances which are particularly suitable for this type of application are known to lack tenacity on laundry, or do not remain on the laundry when rinsed, with the result that their perfuming effect is experienced only briefly and not very intensely. Given the importance of this type of application in the perfume industry, research in this field has been sustained, in particular with the aim of finding new, and more effective solutions to the aforementioned problems.

Delivery systems have been developed in this regards such as microcapsules comprising in their core a perfume being released in a control manner. Microcapsules are particularly relevant to provide the highly sought long lasting perfuming effect leading to benefit from the perfume even after several days.

There is still a need to develop alternative system able to deliver a long lasting perfuming effect up to several days.

It has now been surprisingly found that the invention's composition of matter solves the above-mentioned problem.

### DESCRIPTION OF THE INVENTION

It, has now been discovered that the invention's perfuming composition of matter provides a long lasting effect of several days even a month equal or even better compared to microcapsules while providing an impact directly after use.

So a first object of the present invention is a perfuming composition of matter comprising
a) at least one compound selected from the group consisting of 2-methyl-3-[4-(2-methyl-2-propanyl)phenyl]propanal, 3-(4-isobutyl-2-methylphenyl)propanal, 9-hydroxy-5,9-dimethyl-4-decenal, 1-(4-isopropyl-1-cyclohexyl)-1-ethanol, 2,2-dimethyl-3-(3-methylphenyl)-1-propanol, 7-hydroxy-3,7-dimethyloctanal, 4-(2-propanyl)cyclohexyl]methanol, 3-(4-methoxyphenyl)-2-methylpropanal, 3-(4-tert-butylphenyl)propanal, 3-(4-isopropylphenyl)-2-methylpropanal, (4E)-4-methyl-5-(4-methylphenyl)-4-pentenal, 3/4-(4-hydroxy-4-methylpentyl)-3-cyclohexene-1-carbaldehyde, tetrahydro-2-isobutyl-4-methyl-4(2H)-pyranol, (2,5-Dimethyl-2,3-dihydro-1H-inden-2-yl)methanol, (+−)-8,8-dimethyl-1,2,3,4,5,6,7,8-octahydro-2-naphthalenecarbaldehyde, (+−)-8,8-dimethyl-1,2,3,5,6,7,8,8a-octahydro-2-naphthalenecarbaldehyde, (+−)-8,8-dimethyl-1,2,3,4,6,7,8,8a-octahydro-2-naphthalenecarbaldehyde, (+−)-2,2-dimethyltricyclo[6.2.1.0^{1,6}]undecan-7-one and a mixture thereof;
b) at least one compound selected from the group consisting of tricyclo[5.2.1.0(2,6)]dec-3-en-8-yl propanoate, tricyclo[5.2.1.0(2,6)]dec-4-en-8-yl propanoate, benzyl propanoate, 2-cyclohexylethyl acetate, cyclohexyl acetate, tricyclo[5.2.1.0^{2,6}]dec-3-en-8-yl acetate, tricyclo[5.2.1.0^{2,6}]dec-4-en-8-yl acetate and a mixture thereof;
c) at least one compound selected from the group consisting of 1-(2RS,3RS)-2,3,8,8-tetramethyl-1,2,3,4,5,6,7,8-octahydronaphthalen-2-yl)ethanone, 1-[(2RS,3RS,8aRS)-2,3,8,8-tetramethyl-1,2,3,5,6,7,8,8a-octahydro-2-naphthalenyl]ethanone, 1-[(2RS,3RS,8aRS)-2,3,8,8-tetramethyl-1,2,3,4,6,7,8,8a-octahydro-2-naphthalenyl]ethanone, 1-[(1RS,2RS)-1,2,8,8-tetramethyl-1,2,3,4,5,6,7,8-octahydro-2-naphthalenyl]ethanone, 1-[(1RS,2RS,8aSR)-1,2,8,8-tetramethyl-1,2,3,5,6,7,8,8a-octahydro-2-naphthalenyl]ethenone, 1-(1,2,3,4,5,6,7,8-octahydro-2,3,8,8,-tetramethyl-2-naphthyl)ethan-1-one, (1-ethoxyethoxy)cyclododecane, (ethoxymethoxy)cyclododecane and a mixture thereof;
d) at least, one compound selected from the group consisting of cyclohexyl 2-hydroxybenzoate, 2-methylbutyl salicylate, pentyl salicylate, 2-phenylethyl 2-hydroxybenzoate, hexyl 2-hydroxybenzoate, benzyl salicylate, (3Z)-3-hexen-1-yl salicylate, (Z)-3-hexen-1-yl benzoate and a mixture thereof;
e) at least one compound selected from the group consisting of cyclohexylidene(phenyl)acetonitrile, dodecanenitrile, 3,7-dimethyl-6-octenenitrile, 2,6-nonadienenitrile, 2-tridecenenitrile, 3-tridecenenitrile 3,7-dimethyl-2,6-nonadienenitrile, 2-phenyl-2-hexenenitrile, 3,12-tridecadienenitrile, 3-methyl-5-phenyl-2-pentenenitrile, 2-methyldecanenitrile, and a mixture thereof;
f) at least one compound selected from the group consisting of (+−)-2-methylundecanal, 2-methyldecanal and a mixture thereof;
g) at least one compound selected from the group consisting of patchouli oil, (1R,3R,6S,7S,8S)-2,2,6,8-tetramethyltricyclo[5.3.1.0^{3,8}]undecan-3-ol and a mixture thereof;
h) at least one compound selected from the group consisting of (+−)-3-(4-methyl-3-penten-1-yl)-3-cyclohexene-1-carbaldehyde, (+−)-4-(4-methyl-3-penten-1-yl)-3-cyclohexene-1-carbaldehyde, 3-methyl-5-phenylpentanal, 4-decenal, 2-decenal and a mixture thereof;
i) at least one compound selected from the group consisting of (2E)-1-[(1RS,2SR)-2,6,6-trimethyl-3-cyclohexen-1-yl]-2-buten-1-one, 1-(2,6,6-trimethyl-2-cyclohexen-1-yl)-2-buten-1-one, 1-(2,6,6-trimethyl-1-cyclohexen-1-yl)-2-buten-1-one, 1-(2,4,4-trimethyl-2-cyclohexen-1-yl)-2-buten-1-one, 1-(2,4,4-trimethyl-1-cyclohexen-1-yl)-2-buten-1-one, 1-(2,2-dimethyl-6-methylenecyclohexyl)-2-buten-1-one and a mixture thereof;
j) at least one compound selected from the group consisting of limonene, 2,4,4,7-tetramethyl-6,8-nonadien-3-one oxime, 2-(4-methyl-3-cyclohexen-1-yl)-2-propanethiol, orange oil and a mixture thereof;
k) at least one compound selected from the group consisting of 2-methoxynaphthalene, methyl n-(2-hexyl-3-phenyl-2-propenylidene)anthranilate, methyl n-(3,5,5-trimethylhexylidene)anthranilate, methyl 2-((2,4-dimethylcyclohex-3-enylidene)methylamino)benzoate, methyl n-(1-octenyl)anthranilate, methyl n-(4-methoxybenzylidene)anthranilate, methyl n[3-(4-tert-butylphenyl)-2-methyl-1-propenyl]anthranilate, methyl 2-({[3/4-(4-hydroxy-4-methylpentyl)-3-cyclohexen-1-ylidene]methyl}amino)benzoate, methyl n-benzylideneanthranilate, methyl n-(2-phenylethylidene)anthranilate, methyl n[3-(1,3-benzodioxol-5-yl)-2-methyl-1-propenyl]anthranilate, methyl n[2-(4-methoxybenzyl)-1-propenyl]anthranilate, methyl n-(7-hydroxy-3,7-dimethyl-1-octenyl)anthranilate, methyl 2-(((E)-3-phenylallylidene)amino)benzoate, methyl 2-(decylideneamino)benzoate, methyl [3/4-(4-hydroxy-4-methylpentyl)-3-cyclohexen-1-yl]methyleneanthranilate, methyl 2-[(7-hydroxy-3,7-dimethyloctylidene)amino]benzoate, methyl (3S)-n-(7-hydroxy-3,7-dimethyloctylidene)anthranilate, methyl n-[2-(4-methoxybenzyl)propylidene]anthranilate, ethyl 2-{[(2,4/3,5-dimethyl-3-cyclohexen-1-yl)methyl]amino}benzoate, ethyl 2-{[(1E)-(4-tert-butylphenyl)methylene]amino}benzoate, methyl n-(octylidene)anthranilate, methyl n-[3-(p-tert-butylphenyl)-2-methylpropylidene]anthranilate, methyl n-[3-(4-isopropyl-1-phenyl)-2-methylpropylidene]anthranilate, 2-ethoxynaphthalene, 1-(5,6,7,8-tetrahydro-2-naphthalenyl)ethanone, 1-(3-methyl-1-benzofuran-2-yl)ethanone, 1-(1-naphthyl)ethanone, 1-(2-naphthyl)ethanone, methyl 2-aminobenzoate, methyl n-(7-hydroxy-3,7-dimethyl-1-octenyl)anthranilate, methyl 2-(methylamino)benzoate and a mixture thereof;
l) at least one compound selected from the group consisting of oxacyclohexadecan-2-one, oxacycloheptadecan-2-one, 1-oxa-12-cyclohexadecen-2-one, 1-oxa-13-cyclohexadecen-2-one and a mixture thereof;
m) at least one compound selected from the group consisting of dodecahydro-3a,6,6,9a-tetramethyl-naphtho[2,1-b]furan or any of its stereoisomers and a mixture thereof;
n) at least one compound selected from the group consisting of 1,1′-oxydibenzene, 4-methyl-2-phenyltetrahydro-2H-pyran, tetrahydro-4-methyl-2-(2-methyl-1-propenyl)-2H-pyran, (+−)-4-methylene-2-phenyltetrahydro-2H-pyran, (+−)-4-methyl-6-phenyl-3,6-dihydro-2H-pyran, (+−)-4-methyl-2-phenyl-3,6-dihydro-2H-pyran and a mixture thereof; and
o) at least one compound selected from the group consisting of 10-undecenal, 9-undecenal, decanal and a mixture thereof.

Under a “perfuming composition” is to be understood a composition that is able to impart a hedonic effect to e.g. a consumer product. In other words, a composition to be considered as being a perfuming composition must be recognized by a skilled person in the art of perfumery as being able to impart or modify the olfactory perception in a positive or pleasant way, and not just as imparting an odor.

In particular, the perfuming composition of matter comprises
a) at least one compound selected from the group consisting of 2-methyl-3-[4-(2-methyl-2-propanyl)phenyl]propanal, 3-(4-isobutyl-2-methylphenyl)propanal, 9-hydroxy-5,9-dimethyl-4-decenal, 1-(4-isopropyl-1-cyclohexyl)-1-ethanol, 2,2-dimethyl-3-(3-methylphenyl)-1-propanol, 7-hydroxy-3,7-dimethyloctanal, 4-(2-propanyl)cyclohexyl]methanol, 3-(4-methoxyphenyl)-2-methylpropanal, 3-(4-tert-butylphenyl)propanal, 3-(4-isopropylphenyl)-2-methylpropanal, (4E)-4-methyl-5-(4-methylphenyl)-4-pentenal, 3/4-(4-hydroxy-4-methylpentyl)-3-cyclohexene-1-carbaldehyde, tetrahydro-2-isobutyl-4-methyl-4(2H)-pyranol, (2,5-Dimethyl-2,3-dihydro-1H-inden-2-yl)methanol, (+−)-8,8-dimethyl-1,2,3,4,5,6,7,8-octahydro-2-naphthalenecarbaldehyde, (+−)-8,8-dimethyl-1,2,3,5,6,7,8,8a-octahydro-2-naphthalenecarbaldehyde, (+−)-8,8-dimethyl-1,2,3,4,6,7,8,8a-octahydro-2-naphthalenecarbaldehyde, (+−)-2,2-dimethyltricyclo[6.2.1.0^{1.6}]undecan-7-one and a mixture thereof;
b) at least two compounds selected from the group consisting of tricyclo[5.2.1.0(2,6)]dec-3-en-8-yl propanoate, tricyclo[5.2.1.0(2,6)]dec-4-en-8-yl propanoate, benzyl propanoate, 2-cyclohexylethyl acetate, cyclohexyl acetate, tricyclo[5.2.1.0^{2,6}]dec-3-en-8-yl acetate, tricyclo[5.2.1.0^{2,6}]dec-4-en-8-yl acetate and a mixture thereof;
c) at least, three compounds selected from the group consisting of 1-((2RS,3RS)-2,3,8,8-tetramethyl-1,2,3,4,5,6,7,8-octahydronaphthalen-2-yl)ethanone, 1-[(2RS,3RS,8aRS)-2,3,8,8-tetramethyl-1,2,3,5,6,7,8,8a-octahydro-2-naphthalenyl]ethanone, 1-[(2RS,3RS,8aRS)-2,3,8,8-tetramethyl-1,2,3,4,6,7,8,8a-octahydro-2-naphthalenyl]ethanone, 1-[(1RS,2RS)-1,2,8,8-tetramethyl-1,2,3,4,5,6,7,8-octahydro-2-naphthalenyl]ethanone, 1-[(1RS,2RS,8aSR)-1,2,8,8-tetramethyl-1,2,3,5,6,7,8,8a-octahydro-2-naphthalenyl]ethenone, 1-(1,2,3,4,5,6,7,8-octahydro-2,3,8,8,-tetramethyl-2-naphthyl)ethan-1-one, (1-ethoxyethoxy)cyclododecane, (ethoxymethoxy)cyclododecane and a mixture thereof
d) at, least three compounds selected from the group consisting of cyclohexyl 2-hydroxybenzoate, 2-methylbutyl salicylate, pentyl salicylate, 2-phenylethyl 2-hydroxybenzoate, hexyl 2-hydroxybenzoate, benzyl salicylate, (3Z)-3-hexen-1-yl salicylate, (Z)-3-hexen-1-yl benzoate and a mixture thereof;
e) at least one compound selected from the group consisting of cyclohexylidene(phenyl)acetonitrile, dodecanenitrile, 3,7-dimethyl-6-octenenitrile, 2,6-nonadienenitrile, 2-tridecenenitrile, 3-tridecenenitrile 3,7-dimethyl-2,6-nonadienenitrile, 2-phenyl-2-hexenenitrile, 3,12-tridecadienenitrile, 3-methyl-5-phenyl-2-pentenenitrile, 2-methyldecanenitrile, and a mixture thereof;
f) at, least one compound selected from the group consisting of (+−)-2-methylundecanal, 2-methyldecanal and a mixture thereof;
g) at least one compound selected from the group consisting of patchouli oil, (1R,3R,6S,7S,8S)-2,2,6,8-tetramethyltricyclo[5.3.1.0^{3,8}]undecan-3-ol and a mixture thereof;
h) at least two compounds selected from the group consisting of (+−)-3-(4-methyl-3-penten-1-yl)-3-cyclohexene-1-carbaldehyde, (+−)-4-(4-methyl-3-penten-1-yl)-3-cyclohexene-1-carbaldehyde, 3-methyl-5-phenylpentanal, 4-decenal, 2-decenal and a mixture thereof;
i) at least one compound selected from the group consisting of (2E)-1-[(1RS,2SR)-2,6,6-trimethyl-3-cyclohexen-1-yl]-2-buten-1-one, 1-(2,6,6-trimethyl-2-cyclohexen-1-yl)-2-buten-1-one, 1-(2,6,6-trimethyl-1-cyclohexen-1-yl)-2-buten-1-one, 1-(2,4,4-trimethyl-2-cyclohexen-1-yl)-2-buten-1-one, 1-(2,4,4-trimethyl-1-cyclohexen-1-yl)-2-buten-1-one, 1-(2,2-dimethyl-6-methylenecyclohexyl)-2-buten-1-one and a mixture thereof;
j) at least two compounds selected from the group consisting of limonene, 2,4,4,7-tetramethyl-6,8-nonadien-3-one oxime, 2-(4-methyl-3-cyclohexen-1-yl)-2-propanethiol, orange oil and a mixture thereof;
k) at least one compound selected from the group consisting of 2-methoxynaphthalene, methyl n-(2-hexyl-3-phenyl-2-propenylidene)anthranilate, methyl n-(3,5,5-trimethylhexylidene)anthranilate, methyl 2-((2,4-dimethylcyclohex-3-enylidene)methylamino)benzoate, methyl n-(1-octenyl)anthranilate, methyl n-(4-methoxybenzylidene)anthranilate, methyl n-[3-(4-tert-butylphenyl)-2-methyl-1-propenyl]anthranilate, methyl 2-({[3/4-(4-hydroxy-4-methylpentyl)-3-cyclohexen-1-ylidene]methyl}amino)benzoate, methyl n-benzylideneanthranilate, methyl n-(2-phenylethylidene)anthranilate, methyl n-[3-(1,3-benzodioxol-5-yl)-2-methyl-1-propenyl]anthranilate, methyl n-[2-(4-methoxybenzyl)-1-propenyl]anthranilate, methyl n-(7-hydroxy-3,7-dimethyl-1-octenyl)anthranilate, methyl 2-(((E)-3-phenylallylidene)amino)benzoate, methyl 2-(decylideneamino)benzoate, methyl [3/4-(4-hydroxy-4-methylpentyl)-3-cyclohexen-1-yl]methyleneanthranilate, methyl 2-[(7-hydroxy-3,7-dimethyloctylidene)amino]benzoate, methyl (3S)-n-(7-hydroxy-3,7-dimethyloctylidene)anthranilate, methyl n-[2-(4-methoxybenzyl)propylidene]anthranilate, ethyl 2-{[(2,4/3,5-dimethyl-3-cyclohexen-1-yl)methyl]amino}benzoate, ethyl 2-{[(1E)-(4-tert-butylphenyl)methylene]amino}benzoate, methyl n-(octylidene)anthranilate, methyl n-[3-(p-tert-butylphenyl)-2-methylpropylidene]anthranilate, methyl n-[3-(4-isopropyl-1-phenyl)-2-methylpropylidene]anthranilate, 2-ethoxynaphthalene, 1-(5,6,7,8-tetrahydro-2-naphthalenyl)ethanone, 1-(3-methyl-1-benzofuran-2-yl)ethanone, 1-(1-naphthyl)ethanone, 1-(2-naphthyl)ethanone, methyl 2-aminobenzoate, methyl n-(7-hydroxy-3,7-dimethyl-1-octenyl)anthranilate, methyl 2-(methylamino)benzoate and a mixture thereof;
l) at, least two or three compounds selected from the group consisting of oxacyclohexadecan-2-one, oxacycloheptadecan-2-one, 1-oxa-12-cyclohexadecen-2-one, 1-oxa-13-cyclohexadecen-2-one and a mixture thereof;
m) at least one compound selected from the group consisting of dodecahydro-3a,6,6,9a-tetramethyl-naphtho[2,1-b]furan or any of its stereoisomers and a mixture thereof;
n) at least one compound selected from the group consisting of 1,1′-oxydibenzene, 4-methyl-2-phenyltetrahydro-2H-pyran, tetrahydro-4-methyl-2-(2-methyl-1-propenyl)-2H-pyran, (+−)-4-methylene-2-phenyltetrahydro-2H-pyran, (+−)-4-methyl-6-phenyl-3,6-dihydro-2H-pyran, (+−)-4-methyl-2-phenyl-3,6-dihydro-2H-pyran and a mixture thereof; and
o) at least two compounds selected from the group consisting of 10-undecenal, 9-undecenal, decanal and a mixture thereof.

According to a particular embodiment, the perfuming composition comprises 2-methyl-3-[4-(2-methyl-2-propanyl)phenyl]propanal, tricyclo[5.2.1.0(2,6)]dec-3-en-8-yl propanoate, tricyclo[5.2.1.0(2,6)]dec-4-en-8-yl propanoate, 1-((2RS,3RS)-2,3,8,8-tetramethyl-1,2,3,4,5,6,7,8-octahydronaphthalen-2-yl)ethanone, 1-[(2RS,3RS,8aRS)-2,3,8,8-tetramethyl-1,2,3,5,6,7,8,8a-octahydro-2-naphthalenyl]ethanone, 1-[(2RS,3RS,8aRS)-2,3,8,8-tetramethyl-1,2,3,4,6,7,8,8a- octahydro-2-naphthalenyl]ethanone, cyclohexyl 2-hydroxybenzoate, hexyl 2-hydroxybenzoate, cyclohexylidene(phenyl)acetonitrile, 1-oxa-12-cyclohexadecen-2-one, 1-oxa-13-cyclohexadecen-2-one, (+−)-2-methylundecanal, (3Z)-3-hexen-1-yl salicylate, patchouli oil, (+−)-3-(4-methyl-3-penten-1-yl)-3-cyclohexene-1-carbaldehyde, (+−)-4-(4-methyl-3-penten-1-yl)-3-cyclohexene-1-carbaldehyde, (2E)-1-[(1RS,2SR)-2,6,6-trimethyl-3-cyclohexen-1-yl]-2-buten-1-one, limonene, 2-methoxynaphthalene, oxacyclohexadecan-2-one, dodecahydro-3a,6,6,9a-tetramethyl-naphtho[2,1-b]furan or any of its stereoisomers, 1,1′-oxydibenzene, orange oil, 10-undecenal and 9-undecenal. In other words, the perfuming composition comprises a mixture of all following ingredients: 2-methyl-3-[4-(2-methyl-2-propanyl)phenyl]propanal, tricyclo[5.2.1.0(2,6)]dec-3-en-8-yl propanoate, tricyclo[5.2.1.0(2,6)]dec-4-en-8-yl propanoate, 1-((2RS,3RS)-2,3,8,8-tetramethyl-1,2,3,4,5,6,7,8-octahydronaphthalen-2-yl)ethanone, 1-[(2RS,3RS,8aRS)-2,3,8,8-tetramethyl-1,2,3,5,6,7,8,8a-octahydro-2-naphthalenyl]ethanone, 1-[(2RS,3RS,8aRS)-2,3,8,8-tetramethyl-1,2,3,4,6,7,8,8a-octahydro-2-naphthalenyl]ethanone, cyclohexyl 2-hydroxybenzoate, hexyl 2-hydroxybenzoate, cyclohexylidene(phenyl)acetonitrile, 1-oxa-12-cyclohexadecen-2-one, 1-oxa-13-cyclohexadecen-2-one, (+−)-2-methylundecanal, (3Z)-3-hexen-1-yl salicylate, patchouli oil, (+−)-3-(4-methyl-3-penten-1-yl)-3-cyclohexene-1-carbaldehyde, (+−)-4-(4-methyl-3-penten-1-yl)-3-cyclohexene-1-carbaldehyde, (2E)-1-[(1RS,2SR)-2,6,6-trimethyl-3-cyclohexen-1-yl]-2-buten-1-one, limonene, 2-methoxynaphthalene, oxacyclohexadecan-2-one, dodecahydro-3a,6,6,9a-tetramethyl-naphtho[2,1-b]furan or any of its stereoisomers, 1,1′-oxydibenzene, orange oil, 10-undecenal and 9-undecenal.

According to another particular embodiment, the perfuming composition comprises 2-methyl-3-[4-(2-methyl-2-propanyl)phenyl]propanal, tricyclo[5.2.1.0(2,6)]dec-3-en-8-yl propanoate, tricyclo[5.2.1.0(2,6)]dec-4-en-8-yl propanoate, 1-((2RS,3RS)-2,3,8,8-tetramethyl-1,2,3,4,5,6,7,8-octahydronaphthalen-2-yl)ethanone, 1-[(2RS,3RS,8aRS)-2,3,8,8-tetramethyl-1,2,3,5,6,7,8,8a-octahydro-2-naphthalenyl]ethanone, 1-[(2RS,3RS,8aRS)-2,3,8,8-tetramethyl-1,2,3,4,6,7,8,8a-octahydro-2-naphthalenyl]ethanone, cyclohexyl 2-hydroxybenzoate, hexyl 2-hydroxybenzoate, cyclohexylidene(phenyl)acetonitrile, 1-oxa-12-cyclohexadecen-2-one, 1-oxa-13-cyclohexadecen-2-one, (+−)-2-methylundecanal, (3Z)-3-hexen-1-yl salicylate, patchouli oil, (+−)-3-(4-methyl-3-penten-1-yl)-3-cyclohexene-1-carbaldehyde, (+−)-4-(4-methyl-3-penten-1-yl)-3-cyclohexene-1-carbaldehyde, (2E)-1-[(1RS,2SR)-2,6,6-trimethyl-3-cyclohexen-1-yl]-2-buten-1-one, limonene, 2-methoxynaphthalene, dodecahydro-3a,6,6,9a-tetramethyl-naphtho[2,1-b]furan or any of its stereoisomers, orange oil, tetrahydro-4-methyl-2-(2-methyl-1-propenyl)-2H-pyran, (1-ethoxyethoxy)cyclododecane, 10-undecenal and 9-undecenal. In other words, the perfuming composition comprises a mixture of all following ingredients: 2-methyl-3-[4-(2-methyl-2-propanyl)phenyl]propanal, tricyclo[5.2.1.0(2,6)]dec-3-en-8-yl propanoate, tricyclo[5.2.1.0(2,6)]dec-4-en-8-yl propanoate, 1-((2RS,3RS)-2,3,8,8-tetramethyl-1,2,3,4,5,6,7,8-octahydronaphthalen-2-yl)ethanone, 1-[(2RS,3RS,8aRS)-2,3,8,8-tetramethyl-1,2,3,5,6,7,8,8a-octahydro-2-naphthalenyl]ethanone, 1-[(2RS,3RS,8aRS)-2,3,8,8-tetramethyl-1,2,3,4,6,7,8,8a-octahydro-2-naphthalenyl]ethanone, cyclohexyl 2-hydroxybenzoate, hexyl 2-hydroxybenzoate, cyclohexylidene(phenyl)acetonitrile, 1-oxa-12-cyclohexadecen-2-one, 1-oxa-13-cyclohexadecen-2-one, (+−)-2-methylundecanal, (3Z)-3-hexen-1-yl salicylate, patchouli oil, (+−)-3-(4-methyl-3-penten-1-yl)-3-cyclohexene-1-carbaldehyde, (+−)-4-(4-methyl-3-penten-1-yl)-3-cyclohexene-1-carbaldehyde, (2E)-1-[(1RS,2SR)-2,6,6-trimethyl-3-cyclohexen-1-yl]-2-buten-1-one, limonene, 2-methoxy naphthalene, dodecahydro-3a,6,6,9a-tetramethyl-naphtho[2,1-b]furan or any of its stereoisomers, orange oil, tetrahydro-4-methyl-2-(2-methyl-1-propenyl)-2H-pyran, (1-ethoxyethoxy)cyclododecane, 10-undecenal and 9-undecenal.

According to another particular embodiment, the perfuming composition comprises 2-methyl-3-[4-(2-methyl-2-propanyl)phenyl]propanal, tricyclo[5.2.1.0^{2,6}]dec-3-en-8-yl acetate, tricyclo[5.2.1.0^{2,6}]dec-4-en-8-yl acetate, cyclohexylidene(phenyl)acetonitrile, 1-oxa-12-cyclohexadecen-2-one, 1-oxa-13-cyclohexadecen-2-one, (+−)-2-methylundecanal, 2-methylbutyl salicylate, pentyl salicylate, benzyl salicylate, (3Z)-3-hexen-1-yl salicylate, patchouli oil, (+−)-3-(4-methyl-3-penten-1-yl)-3-cyclohexene-1-carbaldehyde, (+−)-4-(4-methyl-3-penten-1-yl)-3-cyclohexene-1-carbaldehyde, (2E)-1-[(1RS,2SR)-2,6,6-trimethyl-3-cyclohexen-1-yl]-2-buten-1-one, tetrahydro-4-methyl-2-(2-methyl-1-propenyl)-2H-pyran, 1-[(1RS,2RS)-1,2,8,8-tetramethyl-1,2,3,4,5,6,7,8-octahydro-2-naphthalenyl]ethenone, 1-((2RS,3RS)-2,3,8,8-tetramethyl-1,2,3,4,5,6,7,8-octahydronaphthalen-2-yl)ethenone, 1-[(2RS,3RS,8aRS)-2,3,8,8-tetramethyl-1,2,3,5,6,7,8,8a-octahydro-2-naphthalenyl]ethenone, 1-[(1RS,2RS,8aSR)-1,2,8,8-tetramethyl-1,2,3,5,6,7,8,8a-octahydro-2-naphthalenyl]ethenone, 1-[(2RS,3RS,8aRS)-2,3,8,8-tetramethyl-1,2,3,4,6,7,8,8a-octahydro-2-naphthalenyl]ethenone, limonene, 2-methoxynaphthalene, dodecahydro-3a,6,6,9a-tetramethyl-naphtho[2,1-b]furan or any of its stereoisomers, orange oil, 10-undecenal and 9-undecenal. In other words, the perfuming composition comprises a mixture of all following ingredients: 2-methyl-3-[4-(2-methyl-2-propanyl)phenyl]propanal, tricyclo[5.2.1.0^{2,6}]dec-3-en-8-yl acetate, tricyclo[5.2.1.0^{2,6}]dec-4-en-8-yl acetate, cyclohexylidene(phenyl)acetonitrile, 1-oxa-12-cyclohexadecen-2-one, 1-oxa-13-cyclohexadecen-2-one, (+−)-2-methylundecanal, 2-methylbutyl salicylate, pentyl salicylate, benzyl salicylate, (3Z)-3-hexen-1-yl salicylate, patchouli oil, (+−)-3-(4-methyl-3-penten-1-yl)-3-cyclohexene-1-carbaldehyde, (+−)-4-(4-methyl-3-penten-1-yl)-3-cyclohexene-1-carbaldehyde, (2E)-1-[(1RS,2SR)-2,6,6-trimethyl-3-cyclohexen-1-yl]-2-buten-1-one, tetrahydro-4-methyl-2-(2-methyl-1-propenyl)-2H-pyran, 1-[(1RS,2RS)-1,2,8, 8-tetramethyl-1,2,3,4,5,6,7, 8-octahydro-2-naphthalenyl]ethenone, 1-((2RS,3RS)-2,3,8,8-tetramethyl-1,2,3,4,5,6,7,8-octahydronaphthalen-2-yl)ethenone, 1-[(2RS,3RS,8aRS)-2,3,8,8-tetramethyl-1,2,3,5,6,7,8,8a-octahydro-2-naphthalenyl]ethenone, 1-[(1RS,2RS,8aSR)-1,2,8,8-tetramethyl-1,2,3,5,6,7,8,8a-octahydro-2-naphthalenyl]ethenone, 1-[(2RS,3RS,8aRS)-2,3,8,8-tetramethyl-1,2,3,4,6,7,8,8a-octahydro-2-naphthalenyl]ethenone, limonene, 2-methoxynaphthalene, dodecahydro-3a,6,6,9a-tetramethyl-naphtho[2,1-b]furan or any of its stereoisomers, orange oil, 10-undecenal and 9-undecenal.

The expression “patchouli oil” designates any oil extracted from patchouli, any terpenes fractions of patchouli oil or Clearwood® (origin: Firmenich SA).

The expression “dodecahydro-3a,6,6,9a-tetramethyl-naphtho[2,1-b]furan or any of its stereoisomers” has the normal meaning in the art; i.e. dodecahydro-3a,6,6,9a-tetramethyl-naphtho[2,1-b]furan comprises several stereocenter centers being carbon 3a, carbon 5a, carbon 9a and carbon 9b and each of said stereocenter can have two different stereochemistries (e.g. R or S). So, dodecahydro-3a,6,6,9a-tetramethyl-naphtho[2,1-b]furan may be in the form of a pure enantiomer or in the form of a mixture of enantiomers or diastereoisomers. Dodecahydro-3a,6,6,9a-tetramethyl-naphtho[2,1-b]furan can be in a racemic form or scalemic form. Therefore, dodecahydro-3a,6,6,9a-tetramethyl-naphtho[2,1-b]furan can be one stereoisomers or in the form of a composition of matter comprising, or consisting of, various stereoisomers, for example such as (−)-(3aR,5aS,9aS,9bR)-3a,6,6,9a-tetramethyldodecahydronaphtho[2,1-b]furan or (3aRS,5aSR,9aSR,9bRS)-3a,6,6,9a-tetramethyldodecahydronaphtho[2,1-b] furan.

The above mentioned perfuming ingredients can be added to the perfuming composition of matter in a large range of concentrations. In particular, the amount of each perfuming ingredient may be comprised in the range between 0.01 to 30 wt %, preferably between 0.1 to 20 wt %, relative to the total weight of the perfuming composition of matter. In particular, the amount of 2-methyl-3-[4-(2-methyl-2-propanyl)phenyl]propanal may be comprised in the range between 5 to 30 wt %, preferably between 10 to 20 wt %, relative to the total weight of the perfuming composition of matter. In particular, the amount of tricyclo[5.2.1.0(2,6)]dec-3-en-8-yl propanoate and/or tricyclo[5.2.1.0(2,6)]dec-4-en-8-yl propanoate may be comprised in the range between 0.5 to 15 wt %, preferably between 1 to 10 wt %, relative to the total weight of the perfuming composition of matter. In particular, the amount of a mixture comprising 1-((2RS,3RS)-2,3,8,8-tetramethyl-1,2,3,4,5,6,7,8-octahydronaphthalen-2-yl)ethanone, 1-[(2RS,3RS,8aRS)-2,3,8,8-tetramethyl-1,2,3,5,6,7,8,8a-octahydro-2-naphthalenyl]ethanone and/or 1-[(2RS,3RS,8aRS)-2,3,8,8-tetramethyl-1,2,3,4,6,7,8,8a-octahydro-2-naphthalenyl]ethanone may be comprised in the range between 0.5 to 15 wt %, preferably between 1 to 10 wt %, relative to the total weight of the perfuming composition of matter. In particular, the amount of a mixture comprising tricyclo[5.2.1.0^{2,6}]dec-3-en-8-yl acetate and/or tricyclo[5.2.1.0^{2,6}]dec-4-en-8-yl acetate may be comprised in the range between 0.5 to 15 wt %, preferably between 1 to 10 wt %, relative to the total weight of the perfuming composition of matter. In particular, the amount of a mixture comprising 2-methylbutyl salicylate and/or pentyl salicylate may be comprised in the range between 0.5 to 15 wt %, preferably between 1 to 10 wt %, relative to the total weight of the perfuming composition of matter. In particular, the amount of benzyl salicyclate may be comprised in the range between 0.5 to 15 wt %, preferably between 1 to 10 wt %, relative to the total weight of the perfuming composition of matter. In particular, the amount of (1-ethoxyethoxy)cyclododecane may be comprised in the range between 0.1 to 5 wt %, preferably between 0.3 to 2 wt %, relative to the total weight of the perfuming composition of matter. In particular, the amount of tetrahydro-4-methyl-2-(2-methyl-1-propenyl)-2H-pyran may be comprised in the range between 0.1 to 5 wt %, preferably between 0.3 to 2 wt %, relative to the total weight of the perfuming composition of matter. In particular, the amount of cyclohexyl 2-hydroxybenzoate may be comprised in the range between 0.5 to 15 wt %, preferably between 1 to 10 wt %, relative to the total weight of the perfuming composition of matter. In particular, the amount of hexyl 2-hydroxybenzoate may be comprised in the range between 0.5 to 15 wt %, preferably between 1 to 10 wt %, relative to the total weight of the perfuming composition of matter. In particular, the amount of cyclohexylidene(phenyl)acetonitrile may be comprised in the range between 0.5 to 15 wt %, preferably between 1 to 10 wt %, relative to the total weight of the perfuming composition of matter. In particular, the amount of 1-oxa-12-cyclohexadecen-2-one and/or 1-oxa-13-cyclohexadecen-2-one may be comprised in the range between 0.5 to 15 wt %, preferably between 1 to 10 wt %, relative to the total weight of the perfuming composition of matter. In particular, the amount of (+−)-2-methylundecanal may be comprised in the range between 0.5 to 15 wt %, preferably between 1 to 10 wt %, relative to the total weight of the perfuming composition of matter. In particular, the amount of (3Z)-3-hexen-1-yl salicylate may be comprised in the range between 0.5 to 15 wt %, preferably between 1 to 10 wt %, relative to the total weight of the perfuming composition of matter. In particular, the amount of patchouli oil may be comprised in the range between 0.5 to 15 wt %, preferably between 1 to 10 wt %, relative to the total weight of the perfuming composition of matter. In particular, the amount, of a mixture comprising (+−)-3-(4-methyl-3-penten-1-yl)-3-cyclohexene-1-carbaldehyde and/or (+−)-4-(4-methyl-3-penten-1-yl)-3-cyclohexene-1-carbaldehyde may be comprised in the range between 0.5 to 15 wt %, preferably between 1 to 10 wt %, relative to the total weight of the perfuming composition of matter. In particular, the amount of (2E)-1-[(1RS,2SR)-2,6,6-trimethyl-3-cyclohexen-1-yl]-2-buten-1-one may be comprised in the range between 0.5 to 10 wt %, preferably between 1 to 5 wt %, relative to the total weight of the perfuming composition of matter. In particular, the amount of limonene may be comprised in the range between 0.5 to 10 wt %, preferably between 1 to 5 wt %, relative to the total weight of the perfuming composition of matter. In particular, the amount of 2-methoxynaphthalene may be comprised in the range between 0.5 to 10 wt %, preferably between 1 to 5 wt %, relative to the total weight of the perfuming composition of matter. In particular, the amount of oxacyclohexadecan-2-one may be comprised in the range between 0.5 to 10 wt %, preferably between 1 to 5 wt %, relative to the total weight of the perfuming composition of matter. In particular, the amount of dodecahydro-3a,6,6,9a-tetramethyl-naphtho[2,1-b]furan or any of its stereoisomers may be comprised in the range between 0.1 to 5 wt %, preferably between 0.5 to 2 wt %, relative to the total weight of the perfuming composition of matter. In particular, the amount of 1,1′-oxydibenzene may be comprised in the range between 0.1 to 2 wt %, preferably between 0.2 to 1 wt %, relative to the total weight of the perfuming composition of matter. In particular, the amount of orange oil may be comprised in the range between 0.05 to 5 wt %, preferably between 0.1 to 2 wt %, relative to the total weight of the perfuming composition of matter. In particular, the amount of a mixture comprising 10-undecenal and 9-undecenal may be comprised in the range between 0.05 to 1 wt %, preferably between 0.1 to 0.5 wt %, relative to the total weight of the perfuming composition of matter.

Particularly, the invention's perfuming composition of matter may further comprise isopropyl myristate or isopropyl palmitate. The amount, of isopropyl myristate, may be comprised in the range between 0.5 to 10 wt %, preferably between 1 to 5 wt %, relative to the total weight of the perfuming composition.

As mentioned above, the perfuming composition of matter above described shows an increased perception of the perfume over several days. Therefore another object of the present invention is the use as perfuming ingredients of the invention's perfuming composition of matter. In particular, and in other words, a method or a process to confer, enhance, improve or modify the odor properties of a perfuming composition or of a perfumed article or of a surface, which method comprises adding to said composition or article an effective amount, of the perfuming composition of matter according to the invention. For instance, the overall effect imparted by the use of said composition of matter is thus a long lasting effect.

By “use of the perfuming composition of matter” it has to be understood here also the use of any composition containing said composition of matter and which can be advantageously employed in perfumery industry as active ingredients.

Therefore, another object of the present invention is a perfuming composition comprising:
i) as perfuming ingredient, an invention's perfuming composition of matter as defined above;
ii) at least one ingredient selected from the group consisting of a perfumery carrier and a perfumery base; and
iii) optionally at least one perfumery adjuvant.

By “perfumery carrier” it is meant here a material which is practically neutral from a perfumery point of view, i.e. that does not significantly alter the organoleptic properties of perfuming ingredients. Said carrier may be a liquid or a solid.

As liquid carrier one may cite, as non-limiting examples, an emulsifying system, i.e. a solvent and a surfactant system, or a solvent commonly used in perfumery. A detailed description of the nature and type of solvents commonly used in perfumery cannot be exhaustive. However, one can cite as non-limiting examples, solvents such as butylene or propylene glycol, glycerol, dipropyleneglycol and its monoether, 1,2,3-propanetriyl triacetate, dimethyl glutarate, dimethyl adipate 1,3-diacetyloxypropan-2-yl acetate, diethyl phthalate, isopropyl myristate, isopropyl palmitate, benzyl benzoate, benzyl alcohol, 2-(2-ethoxyethoxy)-1-ethano, tri-ethyl citrate or mixtures thereof, which are the most commonly used. For the compositions which comprise both a perfumery carrier and a perfumery base, other suitable perfumery carriers than those previously specified, can be also ethanol, water/ethanol mixtures, terpenes, isoparaffins such as those known under the trademark Isopar (origin: Exxon Chemical) or glycol ethers and glycol ether esters such as those known under the trademark Dowanol (origin: Dow Chemical Company), or hydrogenated castors oils such as those known under the trademark Cremophor RH 40 (origin: BASF).

Solid carrier is meant to designate a material to which the perfuming composition or some element of the perfuming composition can be chemically or physically bound. In general such solid carriers are employed either to stabilize the composition, or to control the rate of evaporation of the compositions or of some ingredients. Solid carriers are of current use in the art and a person skilled in the art knows how to reach the desired effect. However by way of non-limiting examples of solid carriers, one may cite absorbing gums or polymers or inorganic materials, such as porous polymers, cyclodextrins, wood based materials, organic or inorganic gels, clays, gypsum talc or zeolites.

As other non-limiting examples of solid carriers, one may cite encapsulating materials. Examples of such materials may comprise wall-forming and plasticizing materials, such as mono, di- or trisaccharides, natural or modified starches, hydrocolloids, cellulose derivatives, polyvinyl acetates, polyvinylalcohols, proteins or pectins, or yet the materials cited in reference texts such as H. Scherz, Hydrokolloide: Stabilisatoren, Dickungs- and Geliermittel in Lebensmitteln, Band 2 der Schriftenreihe Lebensmittelchemie, Lebensmittelqualität, Behr's Verlag GmbH & Co., Hamburg, 1996. The encapsulation is a well-known process to a person skilled in the art, and may be performed, for instance, by using techniques such as spray-drying, agglomeration or wet extrusion; or consists of a coating encapsulation, including coacervation and complex coacervation techniques.

As non-limiting examples of solid carriers, one may cite in particular the core-shell capsules with resins of aminoplast, polyamide, polyester, polyurea or polyurethane type or a mixture thereof (all of said resins are well known to a person skilled in the art) using techniques like phase separation process induced by polymerization, interfacial polymerization, coacervation or altogether (all of said techniques have been described in the prior art), optionally in the presence of a polymeric stabilizer or of a cationic copolymer.

Resins may be produced by the polycondensation of an aldehyde (e.g. formaldehyde, 2,2-dimethoxyethanal, glyoxal, glyoxylic acid or glycolaldehyde and mixtures thereof) with an amine such as urea, benzoguanamine, glycoluryl, melamine, methylol melamine, methylated methylol melamine, guanazole and the like, as well as mixtures thereof. Alternatively one may use preformed resins alkylolated polyamines such as those commercially available under the trademark Urac® (origin: Cytec Technology Corp.), Cymel® (origin: Cytec Technology Corp.), Urecoll® or Luracoll® (origin: BASF).

Other resins are the ones produced by the polycondensation of an a polyol, like glycerol, and a polyisocyanate, like a trimer of hexamethylene diisocyanate, a trimer of isophorone diisocyanate or xylylene diisocyanate or a Biuret of hexamethylene diisocyanate or a trimer of xylylene diisocyanate with trimethylolpropane (known with the tradename of Takenate®, origin: Mitsui Chemicals), among which a trimer of xylylene diisocyanate with trimethylolpropane and a Biuret of hexamethylene diisocyanate are preferred.

Some of the seminal literature related to the encapsulation of perfumes by polycondensation of amino resins, namely melamine based resins with aldehydes includes articles such as those published by K. Dietrich et al. Acta Polymerica, 1989, vol. 40, pages 243, 325 and 683, as well as 1990, vol. 41, page 91. Such articles already describe the various parameters affecting the preparation of such core-shell microcapsules following prior art methods that are also further detailed and exemplified in the patent literature. U.S. Pat. No. 4,396,670, to the Wiggins Teape Group Limited is a pertinent early example of the latter. Since then, many other authors have enriched the literature in this field and it would be impossible to cover all published developments here, but the general knowledge in encapsulation technology is very significant. More recent publications of pertinence, which disclose suitable uses of such microcapsules, are represented for example by the article of K. Bruyninckx and M. Dusselier, ACS Sustainable Chemistry & Engineering, 2019, vol. 7, pages 8041-8054.

By “perfumery base” what is meant here is a composition comprising at least, one perfuming co-ingredient.

Said perfuming co-ingredient, is not, a perfuming ingredient already present in the invention's composition of matter. Moreover, by “perfuming co-ingredient” it is meant here a compound, which is used in a perfuming preparation or a composition to impart a hedonic effect. In other words such a co-ingredient, to be considered as being a perfuming one, must be recognized by a person skilled in the art as being able to impart or modify in a positive or pleasant way the odor of a composition, and not just as having an odor.

The nature and type of the perfuming co-ingredients present in the base do not warrant a more detailed description here, which in any case would not be exhaustive, the skilled person being able to select them on the basis of his general knowledge and according to the intended use or application and the desired organoleptic effect. In general terms, these perfuming co-ingredients belong to chemical classes as varied as alcohols, lactones, aldehydes, ketones, esters, ethers, acetates, nitriles, terpenoids, nitrogenous or sulphurous heterocyclic compounds and essential oils, and said perfuming co-ingredients can be of natural or synthetic origin.

In particular one may cite perfuming co-ingredients which are commonly used in perfume formulations, such as:
Aldehydic ingredients: decanal, dodecanal, 2-methyl-undecanal, octanal, nonenal and/or nonenal;
Aromatic-herbal ingredients: eucalyptus oil, camphor, eucalyptol, 5-methyltricyclo[6.2.1.0˜2,7˜]undecan-4-one, 1-methoxy-3-hexanethiol, 2-ethyl-4,4-dimethyl-1,3-oxathiane, 2,2,7/8,9/10-Tetramethylspiro[5.5]undec-8-en-1-one, menthol and/or alpha-pinene;
Balsamic ingredients: coumarin, ethylvanillin and/or vanillin;
Citrus ingredients: dihydromyrcenol, citral, linalyl acetate, citronellyl nitrile, orange terpenes, 1-p-menthen-8-yl acetate and/or 1,4(8)-p-menthadiene;
Floral ingredients:methyl dihydrojasmonate, linalool, citronellol, phenylethanol, hexylcinnamic aldehyde, benzyl acetate, benzyl benzoate, beta ionone, methyl (E)-3-methyl-4-(2,6,6-trimethyl-2-cyclohexen-1-yl)-3-buten-2-one, (1E)-1-(2,6,6-trimethyl-2-cyclohexen-1-yl)-1-penten-3-one, 1-(2,6,6-trimethyl-1,3-cyclohexadien-1-yl)-2-buten-1-one, (2E)-1-(2,6,6-trimethyl-2-cyclohexen-1-yl)-2-buten-1-one, (2E)-1-(2,6,6-trimethyl-1-cyclohexen-1-yl)-2-buten-1-one, 2,5-dimethyl-2-indanmethanol, 2,6,6-trimethyl-3-cyclohexene-1-carboxylate, 3-(4,4-dimethyl-1-cyclohexen-1-yl)propanal, 3,7-dimethyl-1,6-nonadien-3-ol, 3-(4-isopropylphenyl)-2-methylpropanal, geraniol, p-menth-1-en-8-ol, 4-(1,1-dimethylethyl)-1-cyclohexyle acetate, 1,1-dimethyl-2-phenylethyl acetate, 4-cyclohexyl-2-methyl-2-butanol, amyl salicylate, high cis methyl dihydrojasmonate, 3-methyl-5-phenyl-1-pentanol, geranyl acetate, tetrahydro linalool, cis-7-p-menthanol, propyl (S)-2-(1,1-dimethylpropoxy)propanoate, 2,2,2-trichloro-1-phenylethyl acetate, amylcinnamic aldehyde, 8-decen-5-olide, 4-phenyl-2-butanone, isononyle acetate, 4-(1,1-dimethylethyl)-1-cyclohexyl acetate, verdyl isobutyrate and/or mixture of methylionones isomers;
Fruity ingredients: gamma-undecalactone, 2,2,5-trimethyl-5-pentylcyclopentanone, 2-methyl-4-propyl-1,3-oxathiane, 4-decanolide, ethyl 2-methyl-pentanoate, hexyl acetate, ethyl 2-methylbutanoate, gamma-nonalactone, allyl heptanoate, 2-phenoxyethyl isobutyrate, ethyl 2-methyl-1,3-dioxolane-2-acetate, 3-(3,3/1,1-dimethyl-5-indanyl)propanal, diethyl 1,4-cyclohexanedicarboxylate, 3-methyl-2-hexen-1-yl acetate, 1-[3,3-dimethylcyclohexyl]ethyl [3-ethyl-2-oxiranyl]acetate and/or diethyl 1,4-cyclohexane dicarboxylate;
Green ingredients: 2-methyl-3-hexanone (E)-oxime, 2,4-dimethyl-3-cyclohexene-1-carbaldehyde, 2-tert-butyl-1-cyclohexyl acetate, styrallyl acetate, allyl (2-methylbutoxy)acetate, 4-methyl-3-decen-5-ol, (Z)-3-hexen-1-ol and/or 1-(5,5-dimethyl-1-cyclohexen-1-yl)-4-penten-1-one;
Musk ingredients: 1,4-dioxa-5,17-cycloheptadecanedione, (Z)-4-cyclopentadecen-1-one, 3-methylcyclopentadecanone, (9Z)-9-cycloheptadecen-1-one, 2-{1S)-1-[(1R)-3,3-dimethylcyclohexyl]ethoxy}-2-oxoethyl propionate 3-methyl-5-cyclopentadecen-1-one, 1,3,4,6,7,8-hexahydro-4,6,6,7,8,8-hexamethyl-cyclopenta-g-2-benzopyrane, (1S,1′R)-2-[1-(3′,3′-dimethyl-1′-cyclohexyl)ethoxy]-2-methylpropyl propanoate and/or (1S,1′R)-[1-(3′,3′-dimethyl-1′-cyclohexyl)ethoxycarbonyl]methyl propanoate;
Woody ingredients: 1-[(1RS,6SR)-2,2,6-trimethylcyclohexyl]-3-hexanol, 3,3-dimethyl-5-[(1R)-2,2,3-trimethyl-3-cyclopenten-1-yl]-4-penten-2- ol, 3,4′-dimethylspiro[oxirane-2,9′-tricyclo[6.2.1.0^{2,7}]undec[4]ene, 2,2,9,11-tetramethylspiro[5.5]undec-8-en-1-yl acetate, (1′R,E)-2-ethyl- 4-(2′,2′,3′-trimethyl-3′-cyclopenten-1′-yl)-2-buten-1-ol, 2-ethyl-4-(2,2,3-trimethyl-3-cyclopenten-1-yl)-2-buten-1-ol, methyl cedryl ketone, 5-(2,2,3-trimethyl-3-cyclopentenyl)-3-methylpentan-2-ol, 1-(2,3,8,8-tetramethyl-1,2,3,4,6,7,8,8a-octahydronaphthalen-2-yl)ethan-1-one and/or isobornyl acetate;
Other ingredients (e.g. amber, powdery spicy or watery): heliotropin, anisic aldehyde, eugenol, cinnamic aldehyde, clove oil, 3-(1,3-benzodioxol-5-yl)-2-methylpropanal, 7-methyl-2H-1,5-benzodioxepin-3(4H)-one, 2,5,5-trimethyl-1,2,3,4,4a,5,6,7-octahydro-2-naphthalenol, 1-phenylvinyl acetate, 6-methyl-7-oxa-1-thia-4-azaspiro[4.4]nonan and/or 3-(3-isopropyl-1-phenyl)butanal.

A perfumery base according to the invention may not be limited to the above mentioned perfuming co-ingredients, and many other of these co-ingredients are in any case listed in reference texts such as the book by S. Arctander, Perfume and Flavor Chemicals, 1969, Montclair, New Jersey, USA, or its more recent versions, or in other works of a similar nature, as well as in the abundant, patent literature in the field of perfumery. It is also understood that said co-ingredients may also be compounds known to release in a controlled manner various types of perfuming compounds also known as properfume or profragrance. Non-limiting examples of suitable properfume may include 3-(dodecylthio)-1-(2,6,6-trimethylcyclohex-3-en-1-yl)butan-1-one (Haloscent® D), 2-(dodecylthio)-4-octanone, 2-(dodecylsulfonyl)octan-4-one, 3-(dodecylthio)-1-(2,6,6-trimethylcyclohex-2-en-1-yl)butan-1-one, 3-(dodecylsulfonyl)-1-(2,6,6-trimethylcyclohex-3-en-1-yl)butan-1-one, 4-(dodecylthio)-4-(2,6,6-trimethylcyclohex-2-en-1-yl)butan-2-one (Haloscent® I) and 4-(dodecylthio)-4-(2,6,6-trimethylcyclohex-1-en-1-yl)butan-2-one (Haloscent® I), a linear polysiloxane co-polymer of 3-((3-(dimethoxy(methyl)silyl)propyl)thio)-2-methyl-5-(prop-1-en-2-yl)cyclohexan-1-one, 4-oxooctan-2-yl dodecanoate, 2-phenylethyl oxo(phenyl)acetate, 3,7-dimethylocta-2,6-dien-1-yl oxo(phenyl)acetate, (Z)-hex-3-en-1-yloxo(phenyl)acetate, 3,7-dimethyl-2,6-octadien-1-yl hexadecanoate, bis(3,7-dimethylocta-2,6-dien-1-yl) succinate, 3-methyl-5-phenylpentyl palmitate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methyl undec-1-ene, (2-((2-methylundec-1-en-1-yl)oxy)ethoxy)benzene, 2-methyl-1-(octan-3-yloxy)undec-1-ene, 1-methoxy-4-(1-phenethoxyprop-1-en-2-yl)benzene, 1-methyl-4-(1-phenethoxyprop-1-en-2-yl)benzene, 2-(1-phenethoxyprop-1-en-2-yl)naphthalene, (2-phenethoxyvinyl)benzene, 2-(1-((3,7-dimethyloct-6-en-1-yl)oxy)prop-1-en-2-yl)naphthalene, 4-allyl-2-methoxy-1-((2-methoxy-2-phenylvinyl)oxy)benzene, (2-((2-pentylcyclopentylidene)methoxy)ethyl)benzene, (2-((2-heptylcyclopentylidene)methoxy)ethyl)benzene, (2-((2-methyl-4-(2,6,6-trimethylcyclohex-1-en-1-yl)but-1-en-1-yl)oxy)ethyl)benzene, 1-methoxy-4-(1-phenethoxyprop-1-en-2-yl)benzene, 1-methoxy-4-(2-methyl-3-phenethoxyallyl)benzene, (2((2-isopropyl-5-methylcyclohexylidene)methoxy)ethyl)benzene, 1-isopropyl-4-methyl-2-((2-pentylcyclopentylidene)methoxy)benzene, 2-methoxy-1-((2-methoxy-2-phenylvinyl)oxy)-4-propylbenzene, 2-ethoxy-1-((2-methoxy-2-phenylvinyl)oxy)-4-methylbenzene, 3-methoxy-4-((2-methoxy-2-phenylvinyl)oxy)benzaldehyde, 1-isopropyl-2-((2-methoxy-2-phenylvinyl)oxy)-4-methylbenzene, 4-((2-(hexyloxy)-2-phenylvinyl)oxy)-3-methoxybenzaldehyde and a mixture thereof. In particular, the invention's perfuming composition comprises at least trans-3-(dodecylthio)-1-(2,6,6-trimethyl-3-cyclohexen-1-yl)-1-butanone and one or more profragrance compounds selected from the group of 4-(dodecylthio)-4-(2,6,6-trimethyl-2-cyclohexen-1-yl)-2-butanone, 4-(dodecylthio)-4-(2,6,6-trimethyl-1-cyclohexen-1-yl)-2-butanone, a linear polysiloxane co-polymer of 3-((3-(dimethoxy(methyl)silyl)propyl)thio)-2-methyl-5-(prop-1-en-2-yl)cyclohexan-1-one, 2-(dodecylthio)octan-4-one, 2-phenylethyl oxo(phenyl)acetate, 3,7-dimethylocta-2,6-dien-1-yl oxo(phenyl)acetate, (Z)-hex-3-en-1-yl oxo(phenyl)acetate, 3,7-dimethyl-2,6-octadien-1-yl hexadecanoate, bis(3,7-dimethylocta-2,6-dien-1-yl) succinate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methylundec-1-ene, (2-((2-methylundec-1-en-1-yl)oxy)ethoxy)benzene, 2-methyl-1-(octan-3-yloxy)undec-1-ene, 1-methoxy-4-(1-phenethoxyprop-1-en-2-yl)benzene, 1-methyl-4-(1-phenethoxyprop-1-en-2-yl)benzene, 2-(1-phenethoxyprop-1-en-2-yl)naphthalene, (2-phenethoxyvinyl)benzene, 2-(1-((3,7-dimethyloct-6-en-1-yl)oxy)prop-1-en-2-yl)naphthalene, 1-(4-(((Z)-hex-3-en-1-yl)oxy)-3-methylbut-3-en-1-yl)-4-methoxybenzene, 4-allyl-2-methoxy-1-((2-methoxy-2-phenylvinyl)oxy)benzene, (2-((2-pentylcyclopentylidene)methoxy)ethyl)benzene, (2-((2-heptylcyclopentylidene)methoxy)ethyl)benzene, (2-((2-pentylcyclopentylidene)methoxy)ethyl)benzene, (2-((2-methyl-4-(2,6,6-trimethylcyclohex-1-en-1-yl)but-1-en-1-yl)oxy)ethyl)benzene, 1-methoxy-4-(1-phenethoxyprop-1-en-2-yl)benzene, 1-methoxy-4-(2-methyl-3-phenethoxyallyl)benzene, (2-((2-isopropyl-5-methylcyclohexylidene)methoxy)ethyl)benzene, 1-isopropyl-4-methyl-2-((2-pentylcyclopentylidene)methoxy)benzene, 2-methoxy-1-((2-methoxy-2-phenylvinyl)oxy)-4-propylbenzene, 2-ethoxy-1-((2-methoxy-2-phenylvinyl)oxy)-4-methylbenzene, 3-methoxy-4-((2-methoxy-2-phenylvinyl)oxy)benzaldehyde, 1-isopropyl-2-((2-methoxy-2-phenylvinyl)oxy)-4-methylbenzene or 4-allyl-2-methoxy-1-((2-methoxy-2-phenylvinyl)oxy)benzene or a mixture thereof.

By “perfumery adjuvant”, it is meant here an ingredient capable of imparting additional added benefit such as a color, a particular light resistance, chemical stability, etc. A detailed description of the nature and type of adjuvant commonly used in perfuming composition cannot be exhaustive, but it has to be mentioned that said ingredients are well known to a person skilled in the art. One may cite as specific non-limiting examples the following: viscosity agents (e.g. surfactants, thickeners, gelling and/or rheology modifiers), stabilizing agents (e.g. preservatives, antioxidant, heat/light and or buffers or chelating agents, such as BHT), coloring agents (e.g. dyes and/or pigments), preservatives (e.g. antibacterial or antimicrobial or antifungal or anti irritant agents), abrasives, skin cooling agents, fixatives, insect repellants, ointments, vitamins and mixtures thereof.

It is understood that a person skilled in the art is perfectly able to design optimal formulations for the desired effect by admixing the above mentioned components of a perfuming composition, simply by applying the standard knowledge of the art, as well as by trial and error methodologies.

An invention's composition consisting of the invention's perfuming composition of matter and at least one perfumery carrier consists of a particular embodiment of the invention as well as a perfuming composition comprising the invention's perfuming composition of matter, at least one perfumery carrier, at least one perfumery base, and optionally at least one perfumery adjuvant.

According to a particular embodiment, the compositions mentioned above, comprise more than one perfuming co-ingredients and enable the perfumer to prepare accords or perfumes possessing the desired odor tonality, creating thus new building block for creation purposes.

For the sake of clarity, it is also understood that any mixture resulting directly from a chemical synthesis, e.g. a reaction medium without an adequate purification, in which the invention's composition would be involved as a starting, intermediate or end-product could not be considered as a perfuming composition according to the invention as far as said mixture does not provide the inventive composition in a suitable form for perfumery. Thus, unpurified reaction mixtures are generally excluded from the present invention unless otherwise specified.

The invention's perfuming composition can also be advantageously used in all the fields of modern perfumery, i.e. fine or functional perfumery, to positively impart or modify the odor of a consumer product into which said perfuming composition is added. Consequently, another object of the present invention consists of by a perfumed consumer product comprising, the invention's perfuming composition.

The invention's composition of matter can be added as such or as part of a perfuming composition.

For the sake of clarity, “perfumed consumer product” is meant to designate a consumer product which delivers at least a pleasant perfuming effect to the surface or space to which it is applied (e.g. skin, hair, textile, paper or home surface) or in the air (air-freshener, deodorizer etc.). In other words, a perfumed consumer product according to the invention is a manufactured product which comprises a functional formulation, as well as optionally additional benefit agents, corresponding to the desired consumer product, and an olfactive effective amount of at least one invention's composition of matter. For the sake of clarity, said perfumed consumer product is a non-edible product.

The nature and type of the constituents of the perfumed consumer product do not warrant a more detailed description here, which in any case would not be exhaustive, the skilled person being able to select them on the basis of his general knowledge and according to the nature and the desired effect of said product.

Non-limiting examples of suitable perfumed consumer products include a perfume, such as a fine perfume, a splash or eau de parfum, a cologne or a shave or after-shave lotion; a fabric care product, such as a liquid, a pod or solid detergent or tablet, a fabric softener, a liquid or solid scent booster, a dryer sheet, a fabric refresher, an ironing water, a paper, a bleach, a carpet cleaner, a curtain-care product; a body-care product, such as a hair care product (e.g. a shampoo, a leave-on or rinse-off hair conditioner, a coloring preparation or a hair spray, a color-care product, a hair shaping product, a dental care product), a disinfectant, an intimate care product; a cosmetic preparation (e.g. a skin cream or lotion, a vanishing cream or a deodorant or antiperspirant (e.g. a spray or roll on), a hair remover, a tanning or sun or after sun product, a nail product, a skin cleansing, a makeup); or a skin-care product (e.g. a soap, a shower or bath mousse, oil or gel, or a hygiene product or a foot/hand care products); an air care product, such as an air freshener or a “ready to use” powdered air freshener which can be used in the home space (rooms, refrigerators, cupboards, shoes or car) and/or in a public space (halls, hotels, malls, etc.); or a home care product, such as a mold remover, a furnisher care product, a wipe, a dish detergent or a hard-surface (e.g. a floor, bath, sanitary or a window-cleaning) detergent; a leather care product; a car care product, such as a polish, a wax or a plastic cleaner. In particular, the perfumed consumer product may be selected from the list consisting of fabric care product, such as a liquid or solid detergent, a fabric softener, a liquid or solid scent booster, a fabric refresher, an ironing water.

Another object of the invention is a perfumed consumer product comprising:
a personal care active base, and
the perfuming composition of matter or the perfuming composition as defined above,
   wherein the consumer product is in the form of a personal care composition.

By “a personal care active base”, it is meant here ingredient(s) capable of imparting a benefit associated with the consumer product such as texture, moisturizing agent, etc . . . The active base comprises ingredients that are common in the perfumed consumer products. The active base of a perfumed consumer product depend on the nature of the perfumed consumer product and can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

The personal care composition is preferably chosen from the group consisting of a hair-care product (e.g. a shampoo, hair conditioner, a coloring preparation or a hair spray), a cosmetic preparation (e.g. a vanishing cream, body lotion or a deodorant or antiperspirant), or a skin-care product (e.g. a perfumed soap, shower or bath mousse, body wash, oil or gel, bath salts, or a hygiene product);

Another object of the invention is a perfumed consumer product comprising:
a home care or a fabric care active base, and
the perfuming composition of matter or the perfuming composition as defined above,
   wherein the consumer product is in the form of a home care or a fabric care composition.

By “a home care or a fabric care active base”, it is meant here ingredient(s) capable of imparting a benefit associated with the consumer product such as texture, moisturizing agent, etc . . . The active base comprises ingredients that are common in the perfumed consumer products. The active base of a perfumed consumer product depend on the nature of the perfumed consumer product and can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

The main functional components of a personal care active base or a home care or a fabric care active bases are surfactants and/or softener components capable of cleaning and/or softening fabrics and/or textiles of varied nature, such as clothes, curtain fabrics, carpet and furniture fabrics, etc, or other home surfaces, skin or hair, and typically used in a large amount of water or water-based solvents. These are therefore formulations wherein the amount of water is typically comprised between 50 and 99% by weight of the active base with the exception of soap or solid detergent wherein the amount of water is at most 20%. The term “base” is used here in the sense of the main component of the composition according to the invention and not in the sense of a liquid having a basic pH.

Preferably, the consumer product comprises from 0.01 to 20 wt %, more preferably between 0.05 and 10 wt % of the perfuming composition of matter of the present invention, these percentages being defined by weight relative to the total weight of the consumer product. Of course, the above concentrations may be adapted according to the benefit effect desired in each product.

An object of the invention is a consumer product in the form of a fabric softener composition comprising:
a fabric softener active base, preferably in an amount comprised between 85 and 99.99% by weight based on the total weight of the composition,
a perfuming composition of matter as defined above, preferably in an amount comprised between 0.01 to 15 wt %, more preferably between 0.05 and 5 wt % by weight based on the total weight of the composition.

The fabric softener active base may comprise dialkyl quaternary ammonium salts, dialkyl ester quaternary ammonium salts (esterquats), Hamburg esterquat (HEQ), triethanolamine quat (TEAQ), cationic guars, silicones and mixtures thereof. Optionally, the fabric softener active base of the composition may further comprise a viscosity modifier in an amount comprised between 0.05 and 1% by weight, based on the total weight of the liquid base; preferably chosen in the group consisting of calcium chloride.

An object, of the invention is a consumer product, in the form of a liquid detergent composition comprising:
a liquid detergent active base, preferably in an amount comprised between 85 and 99.99% by weight based on the total weight of the composition,
a perfuming composition of matter as defined above, preferably in an amount comprised between 0.01 to 15 wt %, more preferably between 0.05 and 5 wt % by weight based on the total weight of the composition.

The liquid detergent active base may comprise anionic surfactant such as alkylbenzenesulfonate (ABS), linear alkylbenzene sulfonates (LAS), secondary alkyl sulfonate (SAS), primary alcohol sulfate (PAS), lauryl ether sulfate (LES), sodium lauryl ether sulfate (SLES), methyl ester sulfonate (MES) and nonionic surfactant such as alkyl amines, alkanolamide, fatty alcohol poly(ethylene glycol) ether, fatty alcohol ethoxylate (FAE), ethylene oxide (EO) and propylene oxide (PO) copolymers, amine oxydes, alkyl polyglucosides, alkyl polyglucosamides.

An object, of the invention is a consumer product, in the form of a solid detergent composition comprising:
a solid detergent active base, preferably in an amount comprised between 85 and 99.95% by weight, based on the total weight, of the composition,
a perfuming composition of matter as defined above, preferably in an amount comprised between 0.05 to 15 wt %, more preferably between 0.1 and 5 wt % by weight based on the total weight, of the composition.

The solid detergent active base may comprises at least one surfactant chosen in the group consisting of anionic, nonionic, cationic, zwiterionic surfactant and mixtures thereof. The surfactant in the solid detergent active base is preferably chosen in the group consisting of linear alkene benzene sulphonate (LABS), sodium laureth sulphate, sodium lauryl ether sulphate (SLES), sodium lauryl sulphate (SLS), alpha olefin sulphonate (AOS), methyl ester sulphonates (MES), alkyl polyglyucosides (APG), primary alcohol ethoxylates and in particular lauryl alcohol ethoxylates (LAE), primary alcohol sulphonates (PAS), soap and mixtures thereof. The soild detergent active base may comprise optionally a further component, commonly used in powder detergent consumer product, selected from the group consisting of bleaching agents such as TAED (tetraacetylethylenediamine); buffering agent; builders such as zeolites, sodium carbonate or mixture thereof; soil release or soil suspension polymers; granulated enzyme particles such as cellulase, lipase, protease, mannanase, pectinase or mixtures thereof; corrosion inhibitor; antifoaming; sud suppressing agents; dyes; fillers such as sodium silicate, sodium sulfate or mixture thereof; source of hydrogen peroxide such as sodium percarbonate or sodium perborate; and mixtures thereof.

An object of the invention is a consumer product in the form of a solid scent booster comprising:
a solid carrier, preferably chosen from the group consisting of urea, sodium chloride, sodium sulphate, sodium acetate, zeolite, sodium carbonate, sodium bicarbonate, clay, talc, calcium carbonate, magnesium sulfate, gypsum, calcium sulfate, magnesium oxide, zinc oxide, titanium dioxide, calcium chloride, potassium chloride, magnesium chloride, zinc chloride, saccharides such as sucrose, mono-, di-, and polysaccharides and derivatives such as starch, cellulose, methyl cellulose, ethyl cellulose, propyl cellulose, polyols/sugar alcohols such as sorbitol, maltitol, xylitol, erythritol, and isomalt, PEG, PVP, citric acid or any water soluble solid acid, fatty alcohols or fatty acids and mixtures thereof;
a perfuming composition of matter, preferably in an amount comprised between 0.05 to 20 wt %, more preferably between 0.1 and 15 wt % by weight based on the total weight of the composition.

An object of the invention is a consumer product in the form of a liquid scent booster comprising:
an aqueous phase,
a surfactant system essentially consisting of one or more than one non-ionic surfactant, wherein the surfactant system has a mean HLB between 10 and 14, preferably chosen from the group consisting of ethoxylated aliphatic alcohols, POE/PPG (polyoxyethylene and polyoxypropylene) ethers, mono and polyglyceryl esters, sucrose ester compounds, polyoxyethylene hydroxylesters, alkyl polyglucosides, amine oxides and combinations thereof;
a linker chosen from the group consisting of alcohols, salts and esters of carboxylic acids, salts and esters of hydroxyl carboxylic acids, fatty acids, fatty acid salts, glycerol fatty acids, surfactant, having an HLB less than 10 and mixtures thereof, and
a perfuming composition of matter as defined above, preferably in an amount comprised between 0.05 to 20 wt %, more preferably between 0.1 and 15 wt % by weight based on the total weight of the composition.

An object of the invention is a consumer product in the form of a shampoo or a shower gel composition comprising:
a shampoo or a shower gel active base, preferably in an amount comprised between 85 and 99.95% by weight based on the total weight of the composition,
a perfuming composition of matter as defined above, preferably in an amount comprised between 0.05 to 15 wt %, more preferably between 0.1 and 5 wt % by weight based on the total weight of the composition.

The shampoo or a shower gel active base may comprise sodium alkylether sulfate, ammonium alkylether sulfates, alkylamphoacetate, cocamidopropyl betaine, cocamide MEA, alkylglucosides and aminoacid based surfactants and mixtures thereof. An object of the invention is a consumer product in the form of a rinse-off conditioner composition comprising:
a rinse-off conditioner active base, preferably in an amount comprised between 85 and 99.95% by weight, based on the total weight, of the composition,
a perfuming composition of matter as defined above, preferably in an amount comprised between 0.05 to 15 wt %, more preferably between 0.1 and 5 wt % by weight based on the total weight of the composition.

The rinse-off conditioner active base may comprise cetyltrimonium chloride, stearyl trimonium chloride, benzalkonium chloride, behentrimonium chloride and mixture thereof.

An object of the invention is a consumer product in the form of an oxidative hair coloring composition comprising:
an oxidizing phase comprising an oxidizing agent and an alkaline phase comprising an alkakine agent, a dye precursor and a coupling compound; wherein said dye precursor and said coupling compound form an oxidative hair dye in the presence of the oxidizing agent, preferably in an amount comprised between 85 and 99.95% by weight based on the total weight of the composition,
a perfuming composition of matter as defined above, preferably in an amount comprised between 0.05 to 15 wt %, more preferably between 0.1 and 5 wt % by weight based on the total weight of the composition.

By “oxidative hair coloring composition”, it is meant a composition comprising two groups of colorless dye molecules: the dye precursor and the coupling agent. Upon reaction with each other through an oxidation process, they form a wide range of colored molecules (dyes) that are then trapped into the hair due their size. In other words, the dye precursor and the coupling compound form an oxidative hair dye in the presence of the oxidizing agent.

“Dye precursor” and “oxidative dye precursor” are used indifferently in the present invention.

Dye precursors can be aromatic compounds derived from benzene substituted by at least two electron donor groups such as NH₂ and OH in para or ortho positions to confer the property of easy oxidation.

According to an embodiment, dye precursors are chosen from the group consisting of p-phenylene diamine, 2,5-diamino toluene, N,N-bis(2-hydroxymethyl)-p-phenylene diamine, 4-aminophenol, 1,4-diamino-benzene, and mixtures thereof.

The primary dye precursor is used in combination with coupling agents. Coupling agents are preferably aromatic compounds derived from benzene and substituted by groups such as NH₂ and OH in the meta position and do not produce color singly, but which modify the color, shade or intensity of the colors developed by the dye precursor.

According to an embodiment, the coupling agent is chosen from the group consisting of resorcinol, 2-methyl resorcinol, 4-chlororesorchinol, 2,5-diamino-toluene, 1,3-diamino-benzene, 2,4-diaminophenoxyethanol HCl, 2-amino-hydroxyethylaminoanisole sulfate, 4-amino-2-hydroxytoluene, and mixtures thereof.

The oxidative dye precursor is preferably used in an amount comprised between 0.001% and 5%, preferably between 0.1% and 4% by weight based on the total weight of the composition.

The use of oxidative dye precursors and coupling agents in hair coloring formulation have been widely disclosed in the prior art and is well-known from the person skilled in the art. One may cite for example EP 0946133A1, the content of which is incorporated by reference.

The alkaline phase comprises an alkaline agent, preferably chosen from the group consisting of ammonia hydroxide, ammonia carbonate, ethanolamine, potassium hydroxide, sodium borate, sodium carbonate, triethanolamine and mixtures thereof.

The alkaline agent is preferably used in an amount comprised between 1% and 10%, preferably between 3% and 9% by weight based on the total weight of the composition.

According to the invention, the coupling agent and the dye precursor in an alkaline medium form an oxidative hair dye in the presence of the oxidizing agent.

The oxidizing agent will supply the necessary oxygen gas to develop color molecules and create a change in hair color.

The oxidizing agent should be safe and effective for use in the compositions herein.

Preferably, the oxidizing agents suitable for use herein will be soluble in the compositions according to the present invention when in liquid form and/or in the form intended to be used.

Preferably, oxidizing agents suitable for use herein will be water-soluble. Suitable oxidizing agents for use herein are selected from inorganic peroxygen oxidizing agents, preformed organic peroxyacid oxidizing agents and organic peroxide oxidizing agents or mixtures thereof.

The oxidizing agent is preferably used in an amount comprised between 5 and 30%, preferably between 5 and 25% by weight based on the total weight of the composition.

Components commonly used in cosmetic compositions may be added into the hair coloring composition as defined in the present invention. One may cite for example, surfactants, cationic polymers, oily substances, silicone derivatives, free perfume, preservatives, ultraviolet absorbents, antioxidants, germicides, propellants, thickeners.

According to a particular embodiment, the hair coloring composition comprises one or more quaternary ammonium compounds, preferably chosen from the group consisting of cetyltrimonium chloride, stearyl trimonium chloride, benzalkonium chloride, behentrimonium chloride and mixture thereof to confer hair conditioner benefits.

Some of the above-mentioned perfumed consumer products may represent an aggressive medium for the invention's perfuming composition of matter, so that it may be necessary to protect the latter from premature decomposition, for example by encapsulation or by chemically binding it to another chemical which is suitable to release the invention's composition or part, of the invention composition upon a suitable external stimulus, such as an enzyme, light, heat or a change of pH.

The proportions in which the compounds according to the invention can be incorporated into the various aforementioned products or compositions vary within a wide range of values. These values are dependent on the nature of the article to be perfumed and on the desired organoleptic effect as well as on the nature of the co-ingredients in a given base when the compounds according to the invention are mixed with perfuming co-ingredients, solvents or additives commonly used in the art.

For example, in the case of perfuming compositions, typical concentrations are in the order of 0.1% to 100% by weight, in particular in the order of 1% to 50%, in particular in the order of 1% to 20%, of the invention's perfuming composition of matter based on the weight of the perfuming composition into which they are incorporated. In the case of perfumed consumer product, typical concentrations are in the order of 0.01% to 20% by weight, or even more, of the invention's perfuming composition of matter based on the weight of the consumer product into which they are incorporated, in particular in the order of 0.05% to 10%.

The invention's perfuming composition of matter is a long lasting perfuming composition. So another object of the invention is a method for prolonging the perception of a perfume on a surface or the air surrounding the perfuming composition, wherein the surface, or the air is treated with the invention's perfuming compositions of matter as defined below. The use as long lasting composition of the invention's perfuming composition of matter as defined below is another object of the invention.

Another object of the invention is a method for conferring, improving, enhancing or modifying fragrance intensity of the fragrance composition on a skin, surface or in the air surrounding of the skin or the surface over a prolonged period of time, preferably over a period of time of more than 72 hours, preferably 120 hours, preferably 250 hours, preferably 500 hours, more preferably 720 hours, under ambient conditions, wherein the method comprises the step of applying the perfuming composition of matter as defined above or the perfuming composition as defined above or the perfumed consumer product as defined above to a skin, surface or air.

For the sake of clarity, by the expression “ambient conditions”, or the similar, it is meant the normal meaning understood by a person skilled in the art, i.e. room temperature, under air and atmospheric pressure.

A further object of the invention is the use of the perfuming composition of matter as defined above or the perfuming composition as defined above or the perfumed consumer product as defined above to improve, enhance or modify the fragrance intensity of the fragrance composition on a skin, surface or in the air surrounding of the skin or the surface over a prolonged period of time, preferably over a period of time of 72 hours, preferably 120 hours, preferably 250 hours, preferably 500 hours, more preferably 720 hours, under ambient conditions

The invention will now be further described by way of examples. It will be appreciated that the invention as claimed is not intended to be limited in any way by these examples.

### EXAMPLES

The invention will now be described in further detail by way of the following examples, wherein the abbreviations have the usual meaning in the art, the temperatures are indicated in degrees centigrade (° C.).

### Example 1

### Preparation of a Invention's Perfuming Composition of Matter and Comparative Perfuming Composition

Perfuming compositions of matter were prepared by admixing the following ingredients:

TABLE 1
Composition of perfuming composition of matter A to C

| | Perfuming composition of matter | | |
|---|---|---|---|
| | Part by Weight | | |
| Ingredients | A¹⁾ | B²⁾ | C¹⁾ |
| (+−)-2-methylundecanal | 400 | | 400 |
| 9/10-undecenal | 20 | | 20 |
| (3aRS,5aSR,9aSR,9bRS)-3a,6,6,9a- | | | 70 |
| tetramethyldodecahydronaphtho[2,1-b]furan and | | | |
| (3aRS,5aSR,9aSR,9bSR)-3a,6,6,9a- | | | |
| tetramethyldodecahydronaphtho[2,1-b]furan | | | |
| 2-methylbutyl salicylate and pentyl salicylate | | | 650 |
| 1-((2RS,3RS)-2,3,8,8-tetramethyl-1,2,3,4,5,6,7,8- | 665 | | |
| octahydronaphthalen-2-yl)ethanone, 1-[(2RS,3RS,8aRS)- | | | |
| 2,3,8,8-tetramethyl-1,2,3,5,6,7,8,8a-octahydro-2- | | | |
| naphthalenyllethanone, 1-[(2RS,3RS,8aRS)-2,3,8,8- | | | |
| tetramethyl-1,2,3,4,6,7,8,8a-octahydro-2-naphthalenyl]ethanone | | | |
| benzyl salicylate | | | 630 |
| (3aRS,5aSR,9aSR,9bRS)-3a,6,6,9a- | 70 | | |
| tetramethyldodecahydronaphtho[2,1-b]furan | | | |
| (3Z)-3-hexen-1-yl salicylate | 400 | 400 | 400 |
| patchouli oil³⁾ | 400 | | |
| cyclohexyl 2-hydroxybenzoate | 650 | 650 | |
| (2E)-1-[(1RS,2SR)-2,6,6-trimethyl-3-cyclohexen-1-yl]-2-buten- | 275 | | 275 |
| 1-one | | | |
| (1-ethoxyethoxy)cyclododecane | 75 | | |
| limonene | 250 | 250 | 250 |
| 1-oxa-12/13-cyclohexadecen-2-one | 600 | | 600 |
| hexyl 2-hydroxybenzoate | 630 | 630 | |
| isopropyl myristate | 200 | 200 | 570 |
| 2-methyl-3-[4-(2-methyl-2-propanyl)phenyl]propanal | 1540 | | 1540 |
| 3/4-(4-methyl-3-penten-1-yl)-3-cyclohexene-1-carbaldehyde | 300 | 300 | 300 |
| orange oil | 25 | 25 | 25 |
| patchouli oil | | | 400 |
| cyclohexylidene(phenyl)acetonitrile | 500 | 500 | 500 |
| tetrahydro-4-methyl-2-(2-methyl-1-propenyl)-2H-pyran | 50 | | 50 |
| 1-[(1RS,2RS)-1,2,8,8-tetramethyl-1,2,3,4,5,6,7,8-octahydro-2- | | | 370 |
| naphthalenyl]ethanone, 1-((2RS,3RS)-2,3,8,8-tetramethyl- | | | |
| 1,2,3,4,5,6,7,8-octahydronaphthalen-2- | | | |
| yl)ethanone,[(2RS,3RS,8aRS)-2,3,8,8-tetramethyl- | | | |
| 1,2,3,5,6,7,8,8a-octahydro-2-naphthalenyl]ethanone, 1- | | | |
| [(1RS,2RS,8aSR)-1,2,8,8-tetramethyl-1,2,3,5,6,7,8,8a- | | | |
| octahydro-2-naphthalenyl]ethanone, 1-[(2RS,3RS,8aRS)- | | | |
| 2,3,8,8-tetramethyl-1,2,3,4,6,7,8,8a-octahydro-2- | | | |
| naphthalenyllethanone | | | |
| tricyclo[5.2.1.0~2,6~]dec-3-en-8-yl acetate and | | | 750 |
| tricyclo[5.2.1.0~2,6~]dec-4-en-8-yl acetate | | | |
| tricyclo[5.2.1.0(2,6)]dec-3-en-8-yl propanoate and | 750 | 750 | |
| tricyclo[5.2.1.0(2,6)]dec-4-en-8-yl propanoate | | | |
| 2-methoxynaphthalene | 200 | | 200 |
| TOTAL | 8000 | 3705 | 8000 |

¹⁾Invention's perfuming composition of matter
²⁾Compartive perfuming composition of matter
³⁾Clearwood ®; origin: Firmenich SA, Geneva, Switzerland

### Example 2

### Performance of a Liquid Detergent Comprising the Invention's Composition of Matter and Comparative Composition of Matter

A model liquid detergent was prepared in a generally known manner with the following composition:

TABLE 2
Composition of the liquid detergent

| Ingredients | Concentration [wt %] |
|---|---|
| Sodium C14-17 Alkyl Sec Sulfonate¹⁾ | 7 |
| Fatty acids, C12-18 and C18-unsaturated²⁾ | 7.5 |
| C12/14 fatty alcohol polyglycol ether with 7 | 17 |
| mol EO³⁾ | |
| Triethanolamine | 7.5 |
| Propylene Glycol | 11 |
| Citric acid | 6.5 |
| Potassium Hydroxyde | 9.5 |
| Protease | 0.2 |
| Amylase | 0.2 |
| Mannanase | 0.2 |
| Acrylates/Steareth-20 Methacrylate | 6 |
| structuring Crosspolymer⁴⁾ | |
| Deionized Water | 27.4 |

¹⁾Hostapur SAS 60; Origin: Clariant
²⁾Edenor K 12-18; Origin: Cognis
³⁾Genapol LA 070; Origin: Clariant
⁴⁾Aculyn 88; Origin: Dow Chemical

Three perfumed liquid detergents were prepared by dispersing 0.25 g of perfuming composition of matter A, B or C in the liquid detergent (dosage in application=0.5%). The sample was mixed well and allowed to macerate for 1 day.

Fabrics (1.7 kg of 30*cotton terry towels) were washed at 35° C. in standard American top loader machine. There were dispensed 50 g of liquid detergent at the start of the wash. After the washing cycle was finished the fabrics were tumble-dried for 45 minutes. The odor intensity of the cotton towels were evaluated by a panel of 8 trained panelists at different times as indicated in Table 3. The panelists were asked to rate the odor intensity of the towels on a scale from 0 to 10, 0 corresponding to no odor and 10 corresponding to extremely strong odor. The results obtained are summarized in Table 3.

TABLE 3
Expert panel evaluation in a liquid detergent

| | Overall Perfume Intensity | | |
|---|---|---|---|
| | Wet | Fresh Dry | 4 days Dry |
| Liquid detergent comprising perfuming | 4.33 | 1 | 4 |
| composition A | | | |
| Liquid detergent comprising perfuming | 4 | 2.75 | 3.07 |
| composition B | | | |
| Liquid detergent comprising perfuming | 5.33 | 4.75 | 4.29 |
| composition C | | | |

Olfactive performance on dry fabrics after 4 days was perceived significantly more intense for liquid detergent comprising perfuming composition A and C (invention's composition) compared to perfuming composition B (comparative composition).

### Example 3

### Preparation of a Granulated PEG Base Comprising the Invention's Composition of Matter

TABLE 4
Composition of the granulated PEG base

| | Bead 1 |
|---|---|
| Ingredients | (%) |
| PEG 4000-7500 | 65 |
| Dextrose | 27 |
| Perfume A or B of Example 1 | 8 |

1) DETE-2624 Alkaline Lipase for detergent , powder form, from creative Enzyme

To the PEG base, dextrose were added and the mixture was melted at 80° C. Then perfuming composition of Example were added. The mixture was then pelletized while cooling, by pouring a thin film of the molten mixture onto a flat surface, and cutting it in smaller pieces after solidification.

### Example 4

### Preparation of a Softener Formulation Comprising the Invention's Composition of Matter

A typical unperfumed softener formulation is listed in Table 5. The unperfumed softener was prepared by weighting Methyl bis[ethyl (tallowate)]-2-hydroxyethyl ammonium methyl sulfate which was heated at 65° C. Then, Water and 1,2-benzisothiazolin-3-one were placed in the reactor and were heated at 65° C. under stirring. To the above mixture was added Methyl bis [ethyl (tallowate)]-2-hydroxyethyl ammonium methyl sulfate. The mixture was stirred 15 minutes and CaCl₂ was added.

TABLE 5
Composition of a typical unperfumed softener formulation.

| Ingredients | Amount (% wt) | Function |
|---|---|---|
| Methyl bis[ethyl(tallowate)]- | 12.2 | Softening agent |
| 2-hydroxyethyl ammonium | | |
| methyl sulfate¹⁾ | | |
| Calcium chloride sol 10% | 0.4 | Viscosity modifier |
| 1,2-benzisothiazolin-3-one²⁾ | 0.04 | Preservative |
| Water | 87.36 | |

¹⁾Stepantex ® VL 90A-Stepan
²⁾Proxel® GXL-Arch

The perfumed softener formulation is then obtained by adding, under gentle shaking, a perfumed composition of Example 1 (0.05 to 1.5% by weight relative to the total weight of the unperfumed softener formulation) into the unperfumed softener formulation listed in Table 5.

### Example 5

### Preparation of Transparent Isotropic Shampoo Formulations Comprising Invention's Perfumed Composition

A typical unperfumed transparent isotropic shampoo formulation is listed in Table 6. The unperfumed shampoo formulation is prepared by dispersing Polyquaternium-10 in water. The remaining ingredients of Phase A are mixed separately by addition of one after the other while mixing well after each adjunction. This pre-mix is added to the Polyquaternium-10 dispersion and mixed for another 5 min. Then, the premixed Phase B and the premixed Phase C are added (Monomuls® 90L-12 is heated to melt in Texapon® NSO IS) while agitating. Phase D and Phase E are added while agitating. The pH is adjusted with a citric acid solution to 5.5-6.0.

TABLE 6
Composition of a typical unperfumed transparent
isotropic shampoo formulation.

| Phase | Ingredients | Amount [wt %] |
|---|---|---|
| A | Deionized water | 44.4 |
| | Polyquaternium-10 ⁽¹⁾ | 0.3 |
| | Glycerin 85% ⁽²⁾ | 1.0 |
| | DMDM Hydantoin ⁽³⁾ | 0.2 |
| B | Sodium laureth sulfate ⁽⁴⁾ | 28.0 |
| | Cocamidopropyl betaine ⁽⁵⁾ | 3.2 |
| | Disodium cocoamphodiacetate ⁽⁶⁾ | 4.0 |
| | Ethoxy (20) stearyl alcohol ⁽⁷⁾ | 1.0 |
| C | Sodium laureth sulfate ⁽⁴⁾ | 3.0 |
| | Glyceryl laureate ⁽⁸⁾ | 0.2 |
| D | Deionized water | 1.0 |
| | Sodium methylparaben ⁽⁹⁾ | 0.1 |
| E | Sodium chloride (10% aqueous solution) | 15.0 |
| | Citric acid (10% aqueous solution to pH 5.5-6.0) | q.s. |

⁽¹⁾ Ucare ® Polymer JR-400; origin: Noveon
⁽²⁾ Origin: Brenntag Schweizerhall AG
⁽³⁾ Glydant ®; origin: Lonza
⁽⁴⁾ Texapon ® NSO IS; origin: Cognis
⁽⁵⁾ Tego ® Betain F 50; origin: Evonik
⁽⁶⁾ Amphotensid GB 2009; origin: Zschimmer & Schwarz
⁽⁷⁾ Brij ® S20; origin: Croda
⁽⁸⁾ Monomuls ® 90 L-12; origin: Gruenau GmbH
⁽⁹⁾ Nipagin Monosodium; origin: NIPA

The perfumed shampoo formulation is then obtained by adding, under gentle shaking, a perfumed composition of Example 1 (0.05 to 1.3% by weight relative to the total weight of the unperfumed shampoo formulation) into the unperfumed shampoo formulation listed in Table 6.

### Example 6

### Preparation of Pearly Shampoo Formulations Comprising Invention's Perfumed Composition

A typical unperfumed pearly shampoo formulation is listed in Table 7. The unperfumed shampoo formulation is prepared by dispersing Tetrasodium EDTA, Guar hydroxypropyltrimonium chloride and Polyquaternium-10 in water. NaOH (10% aqueous solution, Phase B) is added once Phase A is homogeneous. Then, the premixed Phase C is added, and the mixture heated to 75° C. Phase D ingredients are added and mixed until the mixture is homogeneous. The mixture is cooled. At 45° C., Phase E ingredients are added while mixing. The final viscosity is adjusted with NaCl (25% aqueous solution) and a pH of 5.5-6.0 is adjusted with NaOH (10% aqueous solution).

TABLE 7
Composition of a typical pearly shampoo formulation.

| Phase | Ingredients | Amount [wt %] |
|---|---|---|
| A | Deionized water | 45.97 |
| | Tetrasodium EDTA ⁽¹⁾ | 0.05 |
| | Guar hydroxypropyl-trimonium chloride ⁽²⁾ | 0.05 |
| | Polyquaternium-10 ⁽³⁾ | 0.075 |
| B | NaOH (10% aqueous solution) | 0.30 |
| C | Ammonium lauryl sulfate ⁽⁴⁾ | 34.00 |
| | Ammonium laureth sulfate ⁽⁵⁾ | 9.25 |
| | Cocamidopropyl betaine ⁽⁶⁾ | 2.00 |
| | Dimethicone (&) C₁₂₋₁₃ pareth-4 (&) C₁₂₋₁₃ | 2.50 |
| | pareth-23 (&) salicylic acid ⁽⁷⁾ | |
| D | Cetyl alcohol ⁽⁸⁾ | 1.20 |
| | Cocamide MEA ⁽⁹⁾ | 1.50 |
| | Glycol distearate ⁽¹⁰⁾ | 2.00 |
| E | Methylchloroisothiazolinone & | 0.10 |
| | methylisothiazolinone ⁽¹¹⁾ | |
| | D-Panthenol 75% ⁽¹²⁾ | 0.10 |
| | Deionized water | 0.30 |
| F | Sodium chloride (25% aqueous solution) | 0.60 |

⁽¹⁾ EDETA ® B Powder; origin: BASF
⁽²⁾ Jaguar ® C14 S; origin: Rhodia
⁽³⁾ Ucare ® Polymer JR-400; origin: Noveon
⁽⁴⁾ Sulfetal ® LA B-E; origin: Zschimmer & Schwarz
⁽⁵⁾ Zetesol ® LA; origin: Zschimmer & Schwarz
⁽⁶⁾ Tego ® Betain F 50; origin: Evonik
⁽⁷⁾ Xiameter ® MEM-1691; origin: Dow Corning
⁽⁸⁾ Lanette ® 16; origin: BASF
⁽⁹⁾ Comperlan ® 100; origin: Cognis
⁽¹⁰⁾ Cutina ® AGS; origin: Cognis
⁽¹¹⁾ Kathon ® CG; origin: Rohm & Haas
⁽¹²⁾ D-Panthenol; origin: Roche

A perfumed pearly shampoo formulation is then obtained by adding, under gentle shaking, a perfumed composition of Example 1 to 3 (0.05 to 1.3% by weight relative to the total weight of the unperfumed shampoo formulation) into the unperfumed pearly shampoo formulation listed in Table 7.

### Example 7

### Preparation of Rinse-Off Hair Conditioner Formulations Comprising an Invention's Perfumed Composition

A typical unperfumed rinse-off hair conditioner formulation is listed in Table 8. The unperfumed rinse-off hair conditioner formulation is prepared by mixing the ingredients of Phase A until an uniform mixture was obtained. Tylose® is allowed to completely dissolve. Then the mixture is heated to 70-75° C. The ingredients of Phase B are combined and melted at 70-75° C. Then the ingredients of Phase B are added to Phase A with good agitation, and the mixing is continued until that the mixture has a temperature of 60° C. Then, the ingredients of Phase C are added while agitating and keeping mixing until the mixture cooled to 40° C. The pH is adjusted with a citric acid solution to 3.5-4.0.

TABLE 8
Composition of a typical rinse-off hair conditioner formulation.

| | | Amount |
|---|---|---|
| Phase | Ingredients | [wt %] |
| A | Deionized water | 81.8 |
| | Behentrimonium chloride ⁽¹⁾ | 2.5 |
| | Hydroxyethylcellulose ⁽²⁾ | 1.5 |
| B | Cetearyl alcohol ⁽³⁾ | 4.0 |
| | Glyceryl stearate (and) PEG-100 stearate ⁽⁴⁾ | 2.0 |
| | Behentrimonium metho-sulfate (and) cetyl alcohol | 4.0 |
| | (and) butylene glycol ⁽⁵⁾ | |
| | Ethoxy (20) stearyl alcohol ⁽⁶⁾ | 1.0 |
| C | Amodimethicone (and) Trideceth-12 (and) Cetrimonium | 3.0 |
| | chloride ⁽⁷⁾ | |
| | Chlorhexidine digluconate (20% aqueous solution) ⁽⁸⁾ | 0.2 |
| D | Citric acid (10% aqueous solution tol pH 3.5-4.0) | q.s. |

⁽¹⁾ Genamin ® KDMP; origin: Clariant
⁽²⁾ Tylose ® H10 Y G4; origin: Shin Etsu
⁽³⁾ Lanette ® O; origin: BASF
⁽⁴⁾ Arlacel ® 165; origin: Croda
⁽⁵⁾ Incroquat ® Behenyl TMS-50-PA- (MH); origin: Croda
⁽⁶⁾ Brij ® S20; origin: Croda
⁽⁷⁾ Xiameter ® MEM-949; origin: Dow Corning
⁽⁸⁾ Origin: Alfa Aesar

A perfumed rinse-off hair conditioner formulation is then obtained by adding, under gentle shaking, a perfumed composition of Example 1 (0.05 to 1.5% by weight relative to the total weight of the unperfumed conditioner formulation) into the unperfumed rinse-off hair conditioner formulation listed in Table 8.

### Example 8

### Preparation of Structured Shower Gel Formulations Comprising an Invention's Perfumed Composition

A typical unperfumed structured shower gel formulation is listed in Table 9. A perfumed structured shower gel is prepared by adding, under gentle shaking, a perfumed composition of Example 1 (0.05 to 2% by weight relative to the total weight of the structured shower gel) into the unperfumed structured shower gel formulation of Table 9.

TABLE 9
Composition of a typical unperfumed
structured shower gel formulation.

| Ingredients | Amount [wt %] |
|---|---|
| Deionized water | 49.35 |
| Tetrasodium EDTA ⁽¹⁾ | 0.05 |
| Acrylates co-polymer ⁽²⁾ | 6.00 |
| Sodium C₁₂₋₁₅ pareth sulfate ⁽³⁾ | 35.00 |
| Sodium hydroxide (20% aqueous solution) | 1.00 |
| Cocamidopropyl betaine ⁽⁴⁾ | 8.00 |
| Methylchloroisothiazolinone and | 0.10 |
| methylisothiazolinone ⁽⁵⁾ | |
| Citric acid (40% aqueous solution) | 0.50 |

⁽¹⁾ EDETA B powder; origin: BASF
⁽²⁾ Carbopol Aqua SF-1 polymer; origin: Noveon
⁽³⁾ Zetesol AO 328 U; origin: Zschimmer & Schwarz
⁽⁴⁾ Tego Betain F 50; origin: Goldschmidt
⁽⁵⁾ Kathon ® CG; origin: Rohm & Haas

### Example 9

Preparation of Transparent Shower Gel Formulations Comprising an Invention's Perfumed Composition

A typical unperfumed transparent shower gel formulation is listed in Table 10. A perfumed transparent shower gel is prepared by adding, under gentle shaking, a perfumed composition of Example 1 (0.05 to 2% by weight relative to the total weight of the transparent shower gel) into the unperfumed transparent shower gel formulation of Table 10.

TABLE 10
Composition of a typical unperfumed
transparent shower gel formulation

| Ingredients | Amount [wt %] |
|---|---|
| Deionized water | 52.40 |
| Tetrasodium EDTA ⁽¹⁾ | 0.10 |
| Sodium benzoate | 0.50 |
| Propylene glycol | 2.00 |
| Sodium C₁₂₋₁₅ pareth sulfate ⁽²⁾ | 35.00 |
| Cocamidopropyl betaine ⁽³⁾ | 8.00 |
| Polyquaternium-7 ⁽⁴⁾ | 0.20 |
| Citric acid (40% aqueous solution) | 1.00 |
| Sodium chloride | 0.80 |

⁽¹⁾ EDETA B powder; origin: BASF
⁽²⁾ Zetesol AO 328 U; origin: Zschimmer & Schwarz
⁽³⁾ Tego Betain F 50; origin: Goldschmidt
⁽⁴⁾ Merquat ® 550; origin: Lubrizol

### Example 10

### Preparation of Milky Shower Gel Formulations Comprising an Invention's Perfumed Composition

A typical unperfumed milky shower gel formulation is listed in Table 11. A perfumed milky shower gel is prepared by adding, under gentle shaking, a perfumed composition of Example 1 (0.05 to 2% by weight relative to the total weight of the milky shower gel) into the unperfumed milky shower gel formulation of Table 11.

TABLE 11
Composition of a typical unperfumed
milky shower gel formulation.

| Ingredients | Amount [wt %] |
|---|---|
| Deionized water | 50.95 |
| Tetrasodium EDTA ⁽¹⁾ | 0.05 |
| Sodium benzoate | 0.50 |
| Glycerin (86% aqueous solution) | 3.50 |
| Sodium laureth sulfate ⁽²⁾ | 27.00 |
| Polyquaternium-7 ⁽³⁾ | 1.00 |
| Coco-betaine ⁽⁴⁾ | 6.00 |
| PEG-120 Methyl glucose trioleate ⁽⁵⁾ | 1.00 |
| Citric acid (40% aqueous solution) | 1.00 |
| Glycol distearate & laureth-4 & cocamidopropyl | 3.00 |
| betaine ⁽⁶⁾ | |
| Sodium chloride (20% aqueous solution) | 5.00 |
| PEG-40 hydrogenated castor oil ⁽⁷⁾ | 1.00 |

⁽¹⁾ EDETA ® B powder; origin: BASF
⁽²⁾ Texapon ® NSO IS; origin: Cognis
⁽³⁾ Merquat ® 550; origin: Lubrizol
⁽⁴⁾ Dehyton ® AB-30; origin: Cognis
⁽⁵⁾ Glucamate ® LT; origin: Lubrizol
⁽⁶⁾ Euperlan ® PK 3000 AM; origin: Cognis
⁽⁷⁾ Cremophor ® RH 40; origin: BASF

### Example 11

### Preparation of Anhydrous Antiperspirant Spray Formulations Comprising an Invention's Perfumed Composition

A typical unperfumed anhydrous antiperspirant spray formulation is listed in Table 12. The anhydrous antiperspirant spray formulation is prepared by using a high-speed stirrer. Silica and Quaternium-18-hectorite are added to the mixture of isopropyl myristate and cyclomethicone. Once completely swollen, aluminium chlorohydrate is added portion-wise under stirring until the mixture becomes homogeneous and without lumps.

TABLE 12
Composition of a typical unperfumed
anhydrous antiperspirant spray.

| Ingredients | Amount [wt %] |
|---|---|
| Cyclomethicone ⁽¹⁾ | 53.51 |
| Isopropyl myristate | 9.04 |
| Silica ⁽²⁾ | 1.03 |
| Quaternium-18-hectorite ⁽³⁾ | 3.36 |
| Aluminium chlorohydrate ⁽⁴⁾ | 33.06 |

⁽¹⁾ Dow Corning ® 345 Fluid; origin: Dow Corning
⁽²⁾ Aerosil ® 200 ; origin: Evonik
⁽³⁾ Bentone ® 38; origin: Elementis Specialities
⁽⁴⁾ Micro Dry Ultrafine; origin: Reheis

The perfumed formulation is then obtained by adding a perfumed composition of Example (0.05 to 2% by weight relative to the total weight of the antiperspirant spray formulation) into the unperfumed antiperspirant spray formulation of Table 12.

The aerosol cans were filled with 25% Suspension of the suspension and 75% of Propane/Butane (2.5 bar).

### Example 12

### Preparation of Deodorant Spray Emulsion Formulations Comprising an Invention's Perfumed Composition

A typical deodorant spray emulsion formulation is prepared by mixing and dissolving all the ingredients according to the sequence of Table 13. Then a perfumed composition of Example 1 (0.05 to 2% by weight relative to the total weight of the deodorant spray formulation) are added under gentle shaking. Then aerosol cans are filled, and the propellant is crimped and added. Aerosol filling: 40% active solution 60% propane/butane (2.5 bar).

TABLE 13
Composition of a typical unperfumed deodorant spray formulation.

| Ingredients | Amount [wt %] |
|---|---|
| Ethanol (95%) | 90.65 |
| Triclosan ⁽¹⁾ | 0.26 |
| Isopropyl myristate | 9.09 |

⁽¹⁾ Irgasan ® DP 300; origin: BASF

### Example 13

### Preparation of Deodorant Stick Formulations Comprising an Invention's Perfumed Composition

A typical unperfumed deodorant stick formulation is listed in Table 14. The deodorant stick formulation is obtained by weighing all the components of Part A and heating to 70-75° C. Ceteareth-25 is added once the other Part A ingredients are mixed and heated. Once the Ceteareth-25 is dissolved, stearic acid is added. Part B is prepared by dissolving Triclosan in 1,2-propylene glycol. Evaporated water is compensated. Then, slowly, under mixing, Part B is poured into Part A.

TABLE 14
Composition of a typical unperfumed deodorant stick formulation.

| Phase | Ingredients | Amount [wt %] |
|---|---|---|
| A | Stearic acid | 5.05 |
| | 1,2-Propylene glycol | 41.87 |
| | Sodium hydroxide (20% aqueous solution) | 4.24 |
| | Water | 30.30 |
| | Tetrasodium EDTA ⁽¹⁾ | 0.10 |
| | Ceteareth-25 ⁽²⁾ | 1.52 |
| | PPG-3 Myristyl ether ⁽³⁾ | 1.52 |
| B | 1,2-Propylene glycol | 15.14 |
| | Triclosan ⁽⁴⁾ | 0.25 |

⁽¹⁾ Edeta ® B Power; origin: BASF
⁽²⁾ Cremophor ® A25; origin: BASF
⁽³⁾ Tegosoft ® APM; origin: Evonik
⁽⁴⁾ Irgasan ® DP 300; origin: BASF

The perfumed deodorant stick formulation is then obtained by adding a perfumed composition of Example 1 (0.05 to 2% by weight relative to the total weight of the deodorant stick formulation) under gentle shaking. To stock, a plastic bag is put into the bucket to be sealed after cooling. Moulds were filled at about 70° C.

### Example 14

### Preparation of Deodorant Roll-On Formulations Comprising an Invention's Perfumed Composition

A typical unperfumed deodorant roll-on formulation is listed in Table 15. Part A is prepared by sprinkling little-by-little the hydroxyethylcellulose into the water, whilst rapidly stirring with a turbine until the hydroxyethylcellulose is entirely swollen giving a limpid gel. Part B is slowly poured into Part A, whilst continuing stirring until the entire mixture is homogeneous. Then Part C is added.

TABLE 15
Composition of a typical unperfumed
deodorant roll-on formulation.

| Phase | Ingredients | Amount [wt %] |
|---|---|---|
| A | Water | 50.51 |
| | Hydroxyethylcellulose ⁽¹⁾ | 0.71 |
| B | Ethanol (95%) | 40.40 |
| | 1,2-Propylene glycol | 5.05 |
| | Triclosan ⁽²⁾ | 0.30 |
| C | PEG-40 hydrogenated castor oil ⁽³⁾ | 3.03 |

⁽¹⁾ Natrosol ® 250 H; origin: Ashland
⁽²⁾ Irgasan ® DP 300; origin: BASF
⁽³⁾ Cremophor ® RH 40; origin: BASF

The perfumed deodorant, roll-on formulation is then obtained by adding a perfumed composition of Example 1 (0.05 to 2% by weight relative to the total weight of the deodorant, stick formulation) under gentle shaking.

### Example 15

### Preparation of Day Cream Base O/W Emulsions Comprising an Invention's Perfumed Composition

A typical day cream base O/W emulsion formulation is listed in Table 16. Phases A and B are heated separately to 70-75° C., then Phase A is added to Phase B and vacuum is applied. The mixture is stirred and cooled to 55° C. for 15 min. After cooling to room temperature, phenoxyethanol (and) piroctone olamine (Phase C) are added when a temperature of 45° C. is reached. The mixture is stirred for 5 min before sodium carbomer (Phase D), a perfumed composition of Example 1 (Phase E) are added. The mixture is stirred for 3 min, then the stirring was stopped for 15 min. When the temperature of the mixture reaches 30° C., the stirring is resumed for another 15 min until the cream becomes homogeneous, glossy and without lumps. If necessary the pH is adjusted to 6.70-7.20 with Glydant®, Phenoni®p or Nipaguard® PO5 or to 6.30-7.00 with Nikkoguard®.

TABLE 16
Composition of a typical day cream base O/W emulsion.

| Phase | Ingredients | Amount [wt %] |
|---|---|---|
| A | Steareth-2 (and) PEG-8 Distearate⁽¹⁾ | 5.0 |
| | Cetyl alcohol | 0.5 |
| | Ceteth-20 (and) glyceryl stearate (and) | 4.0 |
| | PEG-6 stearate (and) Steareth-20 ⁽²⁾ | |
| | Squalan ⁽³⁾ | 1.0 |
| | Paraffin oil ⁽⁴⁾ | 2.0 |
| | Petrolatum ⁽⁵⁾ | 5.5 |
| B | Deionized water | 75.9 |
| | Propylene glycol | 5.0 |
| C | Phenoxyethanol (and) Piroctone olamine ⁽⁶⁾ | 0.6 |
| D | Sodium carbomer ⁽⁷⁾ | 0.2 |
| E | Perfumed composition of Example 1 | 0.05 to 2 |

⁽¹⁾Arlacel ® 985; origin: Croda
⁽²⁾ Tefose ® 2561; origin: Gattefossé
⁽³⁾ Biolip P 90; origin: Gattefossé
⁽⁴⁾ Mineral oil 30-40 CPS
⁽⁵⁾ Petroleum jelly
⁽⁶⁾ Nipaguard ® PO 5; origin: Clariant
⁽⁷⁾ PNC 400

### Example 16

### Preparation of Hand Dishwash Formulations Comprising an Invention's Perfumed Composition

A typical unperfumed hand dishwash formulation is listed in Table 17. The unperfumed hand dishwash is prepared by mixing water with sodium hydroxide and diethanolamide. Then the linear alkylbenzene sulfonic acid is added. After neutralizing, the remaining ingredients are added and the pH is adjusted to 7-8 if necessary.

TABLE 17
Composition of a typical unperfumed hand dishwash formulation.

| Ingredients | Amount [wt %] |
|---|---|
| Linear alkylbenzene sulfonic acid ⁽¹⁾ | 20.0 |
| Diethanolamide ⁽²⁾ | 3.5 |
| Sodium hydroxide (50%) ⁽³⁾ | 3.4 |
| Secondary alcohol ethoxolate ⁽⁴⁾ | 2.5 |
| Sodium xylene sulfonate | 6.3 |
| Water | 64.3 |

⁽¹⁾ Biosoft ® S-118; origin: Stepan
⁽²⁾ Ninol ® 40-CO; origin: Stepan
⁽³⁾ Stepanate ® SXS; origin: Stepan
⁽⁴⁾ Tergitol ® 15-S-9; origin: Dow Chemicals

The perfumed hand dishwash formulation is then obtained by adding a perfumed composition of Example 1 (0.05 to 2% by weight relative to the total weight of the hand dishwash formulation) under gentle shaking into the unperfumed hand dishwash formulation of Table 17.

### Example 17

### Preparation of an All-Purpose Hard Surface Cleaner Formulations Comprising an Invention's Perfumed Composition

The unperfumed all-purpose hard surface cleaner is prepared by mixing ingredients listed below:

| | |
|---|---|
| Neodol ® 91-8 (origin: Shell Chemicals) | 5.0% by weight |
| Marlon ® A 375 (origin: Hüls AG) | 4.0% by weight |
| Sodium cumolsulphonate | 2.0% by weight |
| Kathon ® CG (origin: Rohm and Haas) | 0.2% by weight |
| Deionized water | 88.8% by weight |

The perfumed all-purpose hard surface cleaner formulation is then obtained by adding a perfumed composition of Example 1 (0.05 to 2% by weight relative to the total weight of the all-purpose hard surface cleaner formulation) under gentle shaking into the unperfumed all-purpose hard surface cleaner formulation mentioned above.

### Example 18

### Preparation of Powder Detergent Formulations Comprising an Invention's Perfumed Composition

A typical unperfumed powder detergent formulation comprises sodium sulfate, sodium carbonate, sodium dodecylbenzensulfonate, sodium silicate, zeolite, C₁₂₋₁₅ pareth-7, bentonite, citric acid, Sodium Acrylic Acid/MA Copolymer, sodium carbonate peroxide, tetrasodium etidronate, sodium chloride, sodium bicarbonate, cellulose gum, Disodium Anilinomorpholinotriazinylaminostilbenesulfonate, Phenylpropyl Dimethicone, enzyme, dye. The base with bleach contained the same as above plus the following additional ingredients: perborate & TAED. The concentrations of each ingredient are indicated in Table 18.

TABLE 18
Typical ranges of ingredients comprised in powder detergent:

| Ingredient | % for bleach formulation |
|---|---|
| Anionic surfactant | 5-25% |
| Non ionic surfactant | 2-15% |
| Builder: | 20-50% |
| Percarborate | 5-20% |
| TAED | 1-8% |
| Polymer | 3-10% |
| Optical brightener | 0.1-0.5% |
| Enzyme, Dye | <2% |

The perfumed powder detergent formulation is then obtained by adding a perfumed composition of Example 1 (0.05 to 2% by weight relative to the total weight of the powder detergent formulation) under gentle shaking into the unperfumed powder detergent as indicated above.

### Example 19

### Preparation of Bleach-Free Powder Detergent Formulations Comprising an Invention's Perfumed Composition

A typical unperfumed bleach-free powder detergent formulation comprises sodium sulfate, sodium carbonate, sodium dodecylbenzensulfonate, sodium silicate, zeolite, C₁₂₋₁₅ pareth-7, bentonite, citric acid, Sodium Acrylic Acid/MA Copolymer, sodium carbonate peroxide, tetrasodium etidronate, sodium chloride, sodium bicarbonate, cellulose gum, Disodium Anilinomorpholinotriazinylaminostilbenesulfonate, Phenylpropyl Dimethicone, enzyme, dye. The concentrations of each ingredient are indicated in Table 19.

TABLE 19
Typical ranges of ingredients comprised in powder detergent:

| Ingredient | % for bleach formulation |
|---|---|
| Anionic surfactant | 5-25% |
| Non ionic surfactant | 2-15% |
| Builder: | 20-50% |
| Percarborate | 5-20% |
| TAED | 1-8% |
| Polymer | 3-10% |
| Optical brightener | 0.1-0.5% |
| Enzyme, Dye | <2% |

The perfumed bleach-free powder detergent formulation is then obtained by adding a perfumed composition of Example 1 (0.05 to 2% by weight relative to the total weight of the bleach-free powder detergent formulation) under gentle shaking into the unperfumed powder detergent as indicated above.

1. A perfuming composition of matter comprising
a) at least one compound selected from the group consisting of 2-methyl-3-[4-(2-methyl-2-propanyl)phenyl]propanal, 3-(4-isobutyl-2-methylphenyl)propanal, 9-hydroxy-5,9-dimethyl-4-decenal, 1-(4-isopropyl-1-cyclohexyl)-1-ethanol, 2,2-dimethyl-3-(3-methylphenyl)-1-propanol, 7-hydroxy-3,7-dimethyloctanal, 4-(2-propanyl)cyclohexyl]methanol, 3-(4-methoxyphenyl)-2-methylpropanal, 3-(4-tert-butylphenyl)propanal, 3-(4-isopropylphenyl)-2-methylpropanal, (4E)-4-methyl-5-(4-methylphenyl)-4-pentenal, 3/4-(4-hydroxy-4-methylpentyl)-3-cyclohexene-1-carbaldehyde, tetrahydro-2-isobutyl-4-methyl-4(2H)-pyranol, (2,5-Dimethyl-2,3-dihydro-1H-inden-2-yl)methanol, (+−)-8,8-dimethyl-1,2,3,4,5,6,7,8-octahydro-2-naphthalenecarbaldehyde, (+−)-8,8-dimethyl-1,2,3,5,6,7,8,8a-octahydro-2-naphthalenecarbaldehyde, (+−)-8,8-dimethyl-1,2,3,4,6,7,8,8a-octahydro-2-naphthalenecarbaldehyde, (+−)-2,2-dimethyltricyclo[6.2.1.0^{1,6}]undecan-7-one and a mixture thereof;
b) at least one compound selected from the group consisting of tricyclo[5.2.1.0(2,6)]dec-3-en-8-yl propanoate, tricyclo[5.2.1.0(2,6)]dec-4-en-8-yl propanoate, benzyl propanoate, 2-cyclohexylethyl acetate, cyclohexyl acetate, tricyclo[5.2.1.0(2,6)]dec-3-en-8-yl acetate, tricyclo[5.2.1.0(2,6)]dec-4-en-8-yl acetate and a mixture thereof;
c) at least one compound selected from the group consisting of 1-((2RS,3RS)-2,3,8,8-tetramethyl-1,2,3,4,5,6,7,8-octahydronaphthalen-2-yl)ethanone, 1-[(2RS,3RS,8aRS)-2,3,8,8-tetramethyl-1,2,3,5,6,7,8,8a-octahydro-2-naphthalenyl]ethanone, 1-[(2RS,3RS,8aRS)-2,3,8,8-tetramethyl-1,2,3,4,6,7,8,8a-octahydro-2-naphthalenyl]ethanone, 1-[(1RS,2RS)-1,2,8,8-tetramethyl-1,2,3,4,5,6,7,8-octahydro-2-naphthalenyl]ethanone, 1-[(1RS,2RS,8aSR)-1,2,8,8-tetramethyl-1,2,3,5,6,7,8,8a-octahydro-2-naphthalenyl]ethenone, 1-(1,2,3,4,5,6,7,8-octahydro-2,3,8,8,-tetramethyl-2-naphthyl)ethan-1-one, (1-ethoxyethoxy)cyclododecane, (ethoxymethoxy)cyclododecane and a mixture thereof;
d) at least one compound selected from the group consisting of cyclohexyl 2-hydroxybenzoate, 2-methylbutyl salicylate, pentyl salicylate, 2-phenylethyl 2-hydroxybenzoate, hexyl 2-hydroxybenzoate, benzyl salicylate, (3Z)-3-hexen-1-yl salicylate, (Z)-3-hexen-1-yl benzoate and a mixture thereof;
e) at least one compound selected from the group consisting of cyclohexylidene(phenyl)acetonitrile, dodecanenitrile, 3,7-dimethyl-6-octenenitrile, 2,6-nonadienenitrile, 2-tridecenenitrile, 3-tridecenenitrile 3,7-dimethyl-2,6-nonadienenitrile, 2-phenyl-2-hexenenitrile, 3,12-tridecadienenitrile, 3-methyl-5-phenyl-2-pentenenitrile, 2-methyldecanenitrile and a mixture thereof;
f) at least one compound selected from the group consisting of (+−)-2-methylundecanal, 2-methyldecanal and a mixture thereof;
g) at least one compound selected from the group consisting of patchouli oil, (1R,3R,6S,7S,8S)-2,2,6,8-tetramethyltricyclo[5.3.1.0^{3,8}]undecan-3-ol and a mixture thereof;
h) at least one compound selected from the group consisting of (+−)-3-(4-methyl-3-penten-1-yl)-3-cyclohexene-1-carbaldehyde, (+−)-4-(4-methyl-3-penten-1-yl)-3-cyclohexene-1-carbaldehyde, 3-methyl-5-phenylpentanal, 4-decenal, 2-decenal and a mixture thereof;
i) at least one compound selected from the group consisting of (2E)-1-[(1RS,2SR)-2,6,6-trimethyl-3-cyclohexen-1-yl]-2-buten-1-one, 1-(2,6,6-trimethyl-2-cyclohexen-1-yl)-2-buten-1-one, 1-(2,6,6-trimethyl-1-cyclohexen-1-yl)-2-buten-1-one, 1-(2,4,4-trimethyl-2-cyclohexen-1-yl)-2-buten-1-one, 1-(2,4,4-trimethyl-1-cyclohexen-1-yl)-2-buten-1-one, 1-(2,2-dimethyl-6-methylenecyclohexyl)-2-buten-1-one and a mixture thereof;
j) at least one compound selected from the group consisting of limonene, 2,4,4,7-tetramethyl-6,8-nonadien-3-one oxime, 2-(4-methyl-3-cyclohexen-1-yl)-2-propanethiol, orange oil and a mixture thereof;
k) at least one compound selected from the group consisting of 2-methoxynaphthalene, methyl n-(2-hexyl-3-phenyl-2-propenylidene)anthranilate, methyl n-(3,5,5-trimethylhexylidene)anthranilate, methyl 2-((2,4-dimethylcyclohex-3-enylidene)methylamino)benzoate, methyl n-(1-octenyl)anthranilate, methyl n-(4-methoxybenzylidene)anthranilate, methyl n-[3-(4-tert-butylphenyl)-2-methyl-1-propenyl]anthranilate, methyl 2-({[3/4-(4-hydroxy-4-methylpentyl)-3-cyclohexen-1-ylidene]methyl}amino)benzoate, methyl n-benzylideneanthranilate, methyl n-(2-phenylethylidene)anthranilate, methyl n-[3-(1,3-benzodioxo1-5-yl)-2-methyl-1-propenyl]anthranilate, methyl n-[2-(4-methoxybenzyl)-1-propenyl]anthranilate, methyl n-(7-hydroxy-3,7-dimethyl-1-octenyl)anthranilate, methyl 2-(((E)-3-phenylallylidene)amino)benzoate, methyl 2-(decylideneamino)benzoate, methyl [3/4-(4-hydroxy-4-methylpentyl)-3-cyclohexen-1-yl]methyleneanthranilate, methyl 2-[(7-hydroxy-3,7-dimethyloctylidene)amino]benzoate, methyl (3S)-n-(7-hydroxy-3,7-dimethyloctylidene)anthranilate, methyl n-[2-(4-methoxybenzyl)propylidene]anthranilate, ethyl 2-{[(2,4/3,5-dimethyl-3-cyclohexen-1-yl)methyl]amino}benzoate, ethyl 2-{[(1E)-(4-tert-butylphenyl)methylene]amino}benzoate, methyl n-(octylidene)anthranilate, methyl n-[3-(p-tert-butylphenyl)-2-methylpropylidene]anthranilate, methyl n-[3-(4-isopropyl-1-phenyl)-2-methylpropylidene]anthranilate, 2-ethoxynaphthalene, 1-(5,6,7,8-tetrahydro-2-naphthalenyl)ethanone, 1-(3-methyl-1-benzofuran-2-yl)ethanone, 1-(1-naphthyl)ethanone, 1-(2-naphthyl)ethanone, methyl 2-aminobenzoate, methyl n-(7-hydroxy-3,7-dimethyl-1-octenyl)anthranilate, methyl 2-(methylamino)benzoate and a mixture thereof;
l) at least one compound selected from the group consisting of oxacyclohexadecan-2-one, oxacycloheptadecan-2-one, 1-oxa-12-cyclohexadecen-2-one, 1-oxa-13-cyclohexadecen-2-one and a mixture thereof;
m) at least one compound selected from the group consisting of dodecahydro-3a,6,6,9a-tetramethyl-naphtho[2,1-b]furan or any of its stereoisomers and a mixture thereof;
n) at least one compound selected from the group consisting of 1,1′-oxydibenzene, 4-methyl-2-phenyltetrahydro-2H-pyran, tetrahydro-4-methyl-2-(2-methyl-1-propenyl)-1H-pyran, (+−)-4-methylene-2-phenyltetrahydro-2H-pyran, (+−)-4-methyl-6-phenyl-3,6-dihydro-2H-pyran, (+−)-4-methyl-2-phenyl-3,6-dihydro-2H-pyran and a mixture thereof; and
o) at least one compound selected from the group consisting of 10-undecenal, 9-undecenal, decanal and a mixture thereof.

2. The perfuming composition according to claim 1, wherein the perfuming composition of matter comprises
a) at least one compound selected from the group consisting of 2-methyl-3-[4-(2-methyl-2-propanyl)phenyl]propanal, 3-(4-isobutyl-2-methylphenyl)propanal, 9-hydroxy-5,9-dimethyl-4-decenal, 1-(4-isopropyl-1-cyclohexyl)-1-ethanol, 2,2-dimethyl-3-(3-methylphenyl)-1-propanol, 7-hydroxy-3,7-dimethyloctanal, 4-(2-propanyl)cyclohexyl]methanol, 3-(4-methoxyphenyl)-2-methylpropanal, 3-(4-tert-butylphenyl)propanal, 3-(4-isopropylphenyl)-2-methylpropanal, (4E)-4-methyl-5-(4-methylphenyl)-4-pentenal, 3/4-(4-hydroxy-4-methylpentyl)-3-cyclohexene-1-carbaldehyde, tetrahydro-2-isobutyl-4-methyl-4(2H)-pyranol, (2,5-Dimethyl-2,3-dihydro-1H-inden-2-yl)methanol, (+−)-8,8-dimethyl-1,2,3,4,5,6,7,8-octahydro-2-naphthalenecarbaldehyde, (+−)-8,8-dimethyl-1,2,3,5,6,7,8,8a-octahydro-2-naphthalenecarbaldehyde, (+−)-8,8-dimethyl-1,2,3,4,6,7,8,8a-octahydro-2-naphthalenecarbaldehyde, (+−)-2,2-dimethyltricyclo[6.2.1.0^{1,6}]undecan-7-one and a mixture thereof;
b) at least two compounds selected from the group consisting of tricyclo[5.2.1.0(2,6)]dec-3-en-8-yl propanoate, tricyclo[5.2.1.0(2,6)]dec-4-en-8-yl propanoate, benzyl propanoate, 2-cyclohexylethyl acetate, cyclohexyl acetate, tricyclo[5.2.1.0^{2,6}]dec-3-en-8-yl acetate, tricyclo[5.2.1.0^{2,6}]dec-4-en-8-yl acetate and a mixture thereof;
c) at least three compounds selected from the group consisting of 1-((2RS,3RS)-2,3,8,8-tetramethyl-1,2,3,4,5,6,7,8-octahydronaphthalen-2-yl)ethanone, 1-[(2RS,3RS,8aRS)-2,3,8,8-tetramethyl-1,2,3,5,6,7,8,8a-octahydro-2-naphthalenyl]ethanone, 1-[(2RS,3RS,8aRS)-2,3,8,8-tetramethyl-1,2,3,4,6,7,8,8a- octahydro-2-naphthalenyl]ethanone, 1-[(1RS,2RS)-1,2,8,8-tetramethyl-1,2,3,4,5,6,7,8-octahydro-2-naphthalenyl]ethanone, 1-[(1RS,2RS,8aSR)-1,2,8,8-tetramethyl-1,2,3,5,6,7,8,8a-octahydro-2-naphthalenyl]ethenone, 1-(1,2,3,4,5,6,7,8-octahydro-2,3,8,8,-tetramethyl-2-naphthyl)ethan-1-one, (1-ethoxyethoxy)cyclododecane, (ethoxymethoxy)cyclododecane and a mixture thereof;
d) at least three compounds selected from the group consisting of cyclohexyl 2-hydroxybenzoate, 2-methylbutyl salicylate, pentyl salicylate, 2-phenylethyl 2-hydroxybenzoate, hexyl 2-hydroxybenzoate, benzyl salicylate, (3Z)-3-hexen-1-yl salicylate, (Z)-3-hexen-1-yl benzoate and a mixture thereof;
e) at least one compound selected from the group consisting of cyclohexylidene(phenyl)acetonitrile, dodecanenitrile, 3,7-dimethyl-6-octenenitrile, 2,6-nonadienenitrile, 2-tridecenenitrile, 3-tridecenenitrile 3,7-dimethyl-2,6-nonadienenitrile, 2-phenyl-2-hexenenitrile, 3,12-tridecadienenitrile, 3-methyl-5-phenyl-2-pentenenitrile, 2-methyldecanenitrile and a mixture thereof;
f) at least one compound selected from the group consisting of (+−)-2-methylundecanal, 2-methyldecanal and a mixture thereof;
g) at least one compound selected from the group consisting of patchouli oil, (1R,3R,6S,7S,8S)-2,2,6,8-tetramethyltricyclo[5.3.1.0^{3,8}]undecan-3-ol and a mixture thereof;
h) at least two compounds selected from the group consisting of (+−)-3-(4-methyl-3-penten-1-yl)-3-cyclohexene-1-carbaldehyde, (+−)-4-(4-methyl-3-penten-1-yl)-3-cyclohexene-1-carbaldehyde, 3-methyl-5-phenylpentanal, 4-decenal, 2-decenal and a mixture thereof;
i) at least one compound selected from the group consisting of (2E)-1-[(1RS,2SR)-2,6,6-trimethyl-3-cyclohexen-1-yl]-2-buten-1-one, 1-(2,6,6-trimethyl-2-cyclohexen-1-yl)-2-buten-1-one, 1-(2,6,6-trimethyl-1-cyclohexen-1-yl)-2-buten-1-one, 1-(2,4,4-trimethyl-2-cyclohexen-1-yl)-2-buten-1-one, 1-(2,4,4-trimethyl-1-cyclohexen-1-yl)-2-buten-1-one, 1-(2,2-dimethyl-6-methylenecyclohexyl)-2-buten-1-one and a mixture thereof;
j) at least two compounds selected from the group consisting of limonene, 2,4,4,7-tetramethyl-6,8-nonadien-3-one oxime, 2-(4-methyl-3-cyclohexen-1-yl)-2-propanethiol, orange oil and a mixture thereof;
k) at least one compound selected from the group consisting of 2-methoxynaphthalene, methyl n-(2-hexyl-3-phenyl-2-propenylidene)anthranilate, methyl n-(3,5,5-trimethylhexylidene)anthranilate, methyl 2-((2,4-dimethylcyclohex-3-enylidene)methylamino)benzoate, methyl n-(1-octenyl)anthranilate, methyl n-(4-methoxybenzylidene)anthranilate, methyl n-[3-(4-tert-butylphenyl)-2-methyl-1-propenyl]anthranilate, methyl 2-({[3/4-(4-hydroxy-4-methylpentyl)-3-cyclohexen-1-ylidene]methyl}amino)benzoate, methyl n-benzylideneanthranilate, methyl n-(2-phenylethylidene)anthranilate, methyl n-[3-(1,3-benzodioxol-5-yl)-2-methyl-1-propenyl]anthranilate, methyl n-[2-(4-methoxybenzyl)-1-propenyl]anthranilate, methyl n-(7-hydroxy-3,7-dimethyl-1-octenyl)anthranilate, methyl 2-(((E)-3-phenylallylidene)amino)benzoate, methyl 2-(decylideneamino)benzoate, methyl [3/4-(4-hydroxy-4-methylpentyl)-3-cyclohexen-1-yl]methyleneanthranilate, methyl 2-[(7-hydroxy-3,7-dimethyloctylidene)amino]benzoate, methyl (3S)-n-(7-hydroxy-3,7-dimethyloctylidene)anthranilate, methyl n-[2-(4-methoxybenzyl)propylidene]anthranilate, ethyl 2-{[(2,4/3,5-dimethyl-3-cyclohexen-1-yl)methyl]amino}benzoate, ethyl 2-{[(1E)-(4-tert-butylphenyl)methylene]amino}benzoate, methyl n-(octylidene)anthranilate, methyl n-[3-(p-tert-butylphenyl)-2-methylpropylidene]anthranilate, methyl n-[3-(4-isopropyl-1-phenyl)-2-methylpropylidene]anthranilate, 2-ethoxynaphthalene, 1-(5,6,7,8-tetrahydro-2-naphthalenyl)ethanone, 1-(3-methyl-1-benzofuran-2-yl)ethanone, 1-(1-naphthyl)ethanone, 1-(2-naphthyl)ethanone, methyl 2-aminobenzoate, methyl n-(7-hydroxy-3,7-dimethyl-1-octenyl)anthranilate, methyl 2-(methylamino)benzoate and a mixture thereof;
l) at least two or three compounds selected from the group consisting of oxacyclohexadecan-2-one, oxacycloheptadecan-2-one, 1-oxa-12-cyclohexadecen-2-one, 1-oxa-13-cyclohexadecen-2-one and a mixture thereof;
m) at least one compound selected from the group consisting of dodecahydro-3a,6,6,9a-tetramethyl-naphtho[2,1-b]furan or any of its stereoisomers and a mixture thereof;
n) at least one compound selected from the group consisting of 1,1′-oxydibenzene, 4-methyl-2-phenyltetrahydro-2H-pyran, tetrahydro-4-methyl-2-(2-methyl-1-propenyl)-2H-pyran, (+−)-4-methylene-2-phenyltetrahydro-2H-pyran, (+−)-4-methyl-6-phenyl-3,6-dihydro-2H-pyran, (+−)-4-methyl-2-phenyl-3,6-dihydro-2H-pyran and a mixture thereof; and
o) at least two compounds selected from the group consisting of 10-undecenal, 9-undecenal, decanal and a mixture thereof.

3. The perfuming composition according to claim 1, wherein the perfuming composition of matter comprises 2-methyl-3-[4-(2-methyl-2-propanyl)phenyl]propanal, tricyclo[5.2.1.0(2,6)]dec-3-en-8-yl propanoate, tricyclo[5.2.1.0(2,6)]dec-4-en-8-yl propanoate, 1-((2RS,3RS)-2,3,8,8-tetramethyl-1,2,3,4,5,6,7,8-octahydronaphthalen-2-yl)ethanone, 1-[(2RS,3RS,8aRS)-2,3,8,8-tetramethyl-1,2,3,5,6,7,8,8a-octahydro-2-naphthalenyl]ethanone, 1-[(2RS,3RS,8aRS)-2,3,8,8-tetramethyl-1,2,3,4,6,7,8,8a-octahydro-2-naphthalenyl]ethanone, cyclohexyl 2-hydroxybenzoate, hexyl 2-hydroxybenzoate, cyclohexylidene(phenyl)acetonitrile, 1-oxa-12-cyclohexadecen-2-one, 1-oxa-13-cyclohexadecen-2-one, (+−)-2-methylundecanal, (3Z)-3-hexen-1-yl salicylate, patchouli oil, (+−)-3-(4-methyl-3-penten-1-yl)-3-cyclohexene-1-carbaldehyde, (+−)-4-(4-methyl-3-penten-1-yl)-3-cyclohexene-1-carbaldehyde, (2E)-1-[(1RS,2SR)-2,6,6-trimethyl-3-cyclohexen-1-yl]-2-buten-1-one, limonene, 2-methoxynaphthalene, dodecahydro-3a,6,6,9a-tetramethyl-naphtho[2,1-b]furan or any of its stereoisomers, orange oil, tetrahydro-4-methyl-2-(2-methyl-1-propenyl)-2H-pyran, (1-ethoxyethoxy)cyclododecane, 10-undecenal and 9-undecenal.

4. The perfuming composition according to claim 1, wherein the perfuming composition of matter further comprises isopropyl myristate or isopropyl palmitate.

5. A perfuming composition comprising:
i) as perfuming ingredient, an invention's perfuming composition of matter as defined in claim 1;
ii) at least one ingredient selected from the group consisting of a perfumery carrier and a perfumery base; and
iii) optionally at least one perfumery adjuvant.

6. The perfuming composition according to claim 5, wherein the perfumery base comprises at least one properfume selected from the group consisting of 3-(dodecylthio)-1-(2,6,6-trimethylcyclohex-3-en-1-yl)butan-1-one, 2-(dodecylthio)-4-octanone, 2-(dodecylsulfonyl)octan-4-one, 3-(dodecylthio)-1-(2,6,6-trimethylcyclohex-2-en-1-yl)butan-1-one, 3-(dodecylsulfonyl)-1-(2,6,6-trimethylcyclohex-3-en-1-yl)butan-1-one, 4-(dodecylthio)-4-(2,6,6-trimethylcyclohex-2-en-1-yl)butan-2-one, 4-(dodecylthio)-4-(2,6,6-trimethylcyclohex-1-en-1-yl)butan-2-one, a linear polysiloxane co-polymer of 3-((3-(dimethoxy(methyl)silyl)propyl)thio)-2-methyl-5-(prop-1-en-2-yl)cyclohexan-1-one, 4-oxooctan-2-yl dodecanoate, 2-phenylethyl oxo(phenyl)acetate, 3,7-dimethylocta-2,6-dien-1-yl oxo(phenyl)acetate, (Z)-hex-3-en-1-yl oxo(phenyl)acetate, 3,7-dimethyl-2,6-octadien-1-yl hexadecanoate, bis(3,7-dimethylocta-2,6-dien-1-yl) succinate, 3-methyl-5-phenylpentyl palmitate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methylundec-1-ene, (2-((2-methylundec-1-en-1-yl)oxy)ethoxy)benzene, 2-methyl-1-(octan-3-yloxy)undec-1-ene, 1-methoxy-4-(1-phenethoxyprop-1-en-2-yl)benzene, 1-methyl-4-(1-phenethoxyprop-1-en-2-yl)benzene, 2-(1-phenethoxyprop-1-en-2-yl)naphthalene, (2-phenethoxyvinyl)benzene, 2-(1-(3,7-dimethyloct-6-en-1-yl)oxy)prop-1-en-2-yl)naphthalene, 4-allyl-2-methoxy-1-((2-methoxy-2-phenylvinyl)oxy)benzene, (2-((2-pentylcyclopentylidene)methoxy)ethyl)benzene, (2-((2-heptylcyclopentylidene)methoxy)ethyl)benzene, (2-((2-methyl-4-(2,6,6-trimethylcyclohex-1-en-1-yl)but-1-en-1-yl)oxy)ethyl)benzene, 1-methoxy-4-(1-phenethoxyprop-1-en-2-yl)benzene, 1-methoxy-4-(2-methyl-3-phenethoxyallyl)benzene, (2-((2-isopropyl-5-methylcyclohexylidene)methoxy)ethyl)benzene, 1-isopropyl-4-methyl-2-((2-pentylcyclopentylidene)methoxy)benzene, 2-methoxy-1-((2-methoxy-2-phenylvinyl)oxy)-4-propylbenzene, 2-ethoxy-1-((2-methoxy-2-phenylvinyl)oxy)-4-methylbenzene, 3-methoxy-4-((2-methoxy-2-phenylvinyl)oxy)benzaldehyde, 1-isopropyl-2-((2-methoxy-2-phenylvinyl)oxy)-4-methylbenzene, 4-((2-(hexyloxy)-2-phenylvinyl)oxy)-3-methoxybenzaldehyde and a mixture thereof.

7. A perfumed consumer product comprising a perfuming composition of matter as defined in claim 1.

8. The perfumed consumer product according to claim 7, characterized in that the perfumery consumer product is a perfume, a fabric care product, a body-care product, a cosmetic preparation, a skin-care product, an air care product or a home care product.

9. The perfumed consumer product according to claim 8, characterized in that the perfumery consumer product is a fine perfume, a splash or eau de parfum, a cologne, a shave or after-shave lotion, a liquid, pod or solid detergent or tablet, a fabric softener, a liquid or solid scent booster, a dryer sheet, a fabric refresher, an ironing water, a paper, a bleach, a carpet cleaner, a curtain-care product, a shampoo, a leave-on or rinse-off hair conditioner, a coloring preparation, a color care product, a hair shaping product, a dental care product, a disinfectant, an intimate care product, a hair spray, a skin cream or lotion, a vanishing cream, a deodorant or antiperspirant, a hair remover, a tanning or sun or after sun product, a nail product, a skin cleansing, a makeup, a perfumed soap, a shower or bath mousse, oil or gel, a foot/hand care product, a hygiene product, an air freshener, a “ready to use” powdered air freshener, a mold remover, a furnisher care, a wipe, a dish detergent or a hard-surface detergent, a leather care product, or a car care product.

10-11. (canceled)

12. A method for conferring, improving, enhancing or modifying fragrance intensity of the fragrance composition on a skin, surface or in the air surrounding of the skin or the surface over a prolonged period of time under ambient conditions, wherein the method comprises the step of applying the perfuming composition of matter as defined in claim 1 to a skin, surface or air.

13. A method to confer, enhance, improve or modify the odor properties of a perfuming composition or of a perfumed article, which method comprises adding to said composition or article an effective amount of the perfuming composition of matter as defined in claim 1.

14. The method according to claim 12, wherein the prolonged period of time is more than 72 hours.

15. A perfuming composition comprising:
i) as perfuming ingredient, a perfuming composition of matter as defined in claim 2;
ii) at least one ingredient selected from the group consisting of a perfumery carrier and a perfumery base; and
iii) optionally at least one perfumery adjuvant.

16. The perfuming composition according to claim 15, wherein the perfumery base comprises at least one properfume selected from the group consisting of 3-(dodecylthio)-1-(2,6,6-trimethylcyclohex-3-en-1-yl)butan-1-one, 2-(dodecylthio)-4-octanone, 2-(dodecylsulfonyl)octan-4-one, 3-(dodecylthio)-1-(2,6,6-trimethylcyclohex-2-en-1-yl)butan-1-one, 3-(dodecyl sulfonyl)-1-(2,6,6-trimethylcyclohex-3-en-1-yl)butan-1-one, 4-(dodecylthio)-4-(2,6,6-trimethylcyclohex-2-en-1-yl)butan-2-one, 4-(dodecylthio)-4-(2,6,6-trimethylcyclohex-1-en-1-yl)butan-2-one, a linear polysiloxane co-polymer of 3-((3-(dimethoxy(methyl)silyl)propyl)thio)-2-methyl-5-(prop-1-en-2-yl)cyclohexan-1-one, 4-oxooctan-2-yl dodecanoate, 2-phenylethyl oxo(phenyl)acetate, 3,7-dimethylocta-2,6-dien-1-yl oxo(phenyl)acetate, (Z)-hex-3-en-1-yl oxo(phenyl)acetate, 3,7-dimethyl-2,6-octadien-1-yl hexadecanoate, bis(3,7-dimethylocta-2,6-dien-1-yl) succinate, 3-methyl-5-phenylpentyl palmitate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methylundec-1-ene, (2-((2-methylundec-1-en-1-yl)oxy)ethoxy)benzene, 2-methyl-1-(octan-3-yloxy)undec-1-ene, 1-methoxy-4-(1-phenethoxyprop-1-en-2-yl)benzene, 1-methyl-4-(1-phenethoxyprop-1-en-2-yl)benzene, 2-(1-phenethoxyprop-1-en-2-yl)naphthalene, (2-phenethoxyvinyl)benzene, 2-(1-((3,7-dimethyloct-6-en-1-yl)oxy)prop-1-en-2-yl)naphthalene, 4-allyl-2-methoxy-1-((2-methoxy-2-phenylvinyl)oxy)benzene, (2-((2-pentylcyclopentylidene)methoxy)ethyl)benzene, (2-((2-heptylcyclopentylidene)methoxy)ethyl)benzene, (2-((2-methyl-4-(2,6,6-trimethylcyclohex-1-en-1-yl)but-1-en-1-yl)oxy)ethyl)benzene, 1-methoxy-4-(1-phenethoxyprop-1-en-2-yl)benzene, 1-methoxy-4-(2-methyl-3-phenethoxyallyl)benzene, (2-((2-isopropyl-5-methylcyclohexylidene)methoxy)ethyl)benzene, 1-isopropyl-4-methyl-2-((2-pentylcyclopentylidene)methoxy)benzene, 2-methoxy-1-((2-methoxy-2-phenylvinyl)oxy)-4-propylbenzene, 2-ethoxy-1-((2-methoxy-2-phenylvinyl)oxy)-4-methylbenzene, 3-methoxy-4-((2-methoxy-2-phenylvinyl)oxy)benzaldehyde, 1-isopropyl-2-((2-methoxy-2-phenylvinyl)oxy)-4-methylbenzene, 4-((2-(hexyloxy)-2-phenylvinyl)oxy)-3-methoxybenzaldehyde and a mixture thereof.

17. A perfumed consumer product comprising a perfuming composition of matter as defined in claim 2.

18. The perfumed consumer product according to claim 17, characterized in that the perfumery consumer product is a perfume, a fabric care product, a body-care product, a cosmetic preparation, a skin-care product, an air care product or a home care product.

19. The perfumed consumer product according to claim 18, characterized in that the perfumery consumer product is a fine perfume, a splash or eau de parfum, a cologne, a shave or after-shave lotion, a liquid, pod or solid detergent or tablet, a fabric softener, a liquid or solid scent booster, a dryer sheet, a fabric refresher, an ironing water, a paper, a bleach, a carpet cleaner, a curtain-care product, a shampoo, a leave-on or rinse-off hair conditioner, a coloring preparation, a color care product, a hair shaping product, a dental care product, a disinfectant, an intimate care product, a hair spray, a skin cream or lotion, a vanishing cream, a deodorant or antiperspirant, a hair remover, a tanning or sun or after sun product, a nail product, a skin cleansing, a makeup, a perfumed soap, a shower or bath mousse, oil or gel, a foot/hand care product, a hygiene product, an air freshener, a “ready to use” powdered air freshener, a mold remover, a furnisher care, a wipe, a dish detergent or a hard-surface detergent, a leather care product, or a car care product.

20. A method for conferring, improving, enhancing or modifying fragrance intensity of the fragrance composition on a skin, surface or in the air surrounding of the skin or the surface over a prolonged period of time under ambient conditions, wherein the method comprises the step of applying the perfuming composition of matter as defined in claim 2 to a skin, surface or air.

21. The method according to claim 20, wherein the prolonged period of time is more than 72 hours.

22. A method to confer, enhance, improve or modify the odor properties of a perfuming composition or of a perfumed article, which method comprises adding to said composition or article an effective amount of the perfuming composition of matter as defined in claim 2.
